(19) United States
(12) Patent Application Publication
(10) Pub. No.: US 2025/0152696 A1
(43) Pub. Date: May 15, 2025

(54) METHODS AND COMPOSITIONS FOR VACCINATION AGAINST HETEROSUBTYPIC INFLUENZA VIRUSES USING AN ADENOVIRAL VECTOR LEADING TO ENHANCED T CELL RESPONSE THROUGH AUTOPHAGY

(71) Applicants: Purdue Research Foundation, West Lafayette, IN (US); The United States of America as represented by the Secretary, Department of Health and Human Service, Bethesda, MD (US); The Methodist Hospital, Houston, TX (US)

(72) Inventors: Suresh MITTAL, West Lafayette, IN (US); Suryaprakash SAMBHARA, Atlanta, GA (US); Ekramy SAYEDAHMED, West Lafayette, IN (US); Chinnaswamy JAGANNATH, Sugar Land, TX (US)

(21) Appl. No.: 18/683,238

(22) PCT Filed: Aug. 13, 2022
(86) PCT No.: PCT/US2022/074946

§ 371 Date: Feb. 12, 2024

Related U.S. Application Data
(60) Provisional application No. 63/232,722, filed on Aug. 13, 2021.

(51) Int. Cl. A61K 39/145; A61K 39/00; A61P 37/04

(52) U.S. Cl. CPC A61K 39/145; A61P 37/04; A61K 2039/54; A61K 2039/542; A61K 2039/543; A61K 2039/6068; A61K 2039/70

(57) ABSTRACT

An adenoviral vector with E1 and E3 regions removed and expressing the nucleoprotein or other immunogenic domain(s) of an influenza virus with or without the presence of the Autophagy-Inducing Peptide C5 (AIP-C5) from the CFP10 protein of Mycobacterium tuberculosis, compositions comprising same, and methods of use for general vaccination against heterosubtypic influenza viruses.

### PRIORITY

This patent application is related to and claims the priority benefit of U.S. Provisional Patent Application No. 63/232,722 filed Aug. 13, 2021. The content of the foregoing application is hereby incorporated by reference in its entirety into this disclosure.

### STATEMENT OF GOVERNMENT SUPPORT

This invention was made with government support under AI059374 awarded by the National Institutes of Health. The government has certain rights in the invention.

### TECHNICAL FIELD

The present disclosure relates to a universal influenza vaccine, methods and compositions for general vaccination against heterosubtypic influenza viruses using a human or bovine adenoviral vector with the E1 and E3 regions removed and expressing the nucleoprotein of influenza virus H7N9 or other immunogenic domain(s) of an influenza virus with or without the presence of the Autophagy-Inducing Peptide C5 from the CFP10 protein of Mycobacterium tuberculosis.

### BACKGROUND

This section introduces aspects that may help facilitate a better understanding of the disclosure. Accordingly, these statements are to be read in this light and are not to be understood as admissions about what is or is not prior art.

Influenza viruses continue to pose a significant threat to human health worldwide. Approximately one billion human infections, 3 to 5 million severe cases, and 300,000 to 500,000 deaths occur every year despite the availability of influenza vaccines. Influenza viruses are known for continuous antigenic changes due to the immune pressure and faulty genome replication system. This antigenic drift lowers the efficacy of seasonal influenza vaccines.

Besides seasonal influenza viruses (e.g., H1N1, H3N2, and influenza B), reports of human infections with either low or highly pathogenic avian influenza (HPAI) A viruses of H5, H7, and H9 subtypes underscore the public health threat and pandemic potential posed by these avian influenza viruses (AIV). Since their emergence in Asia over two decades ago, HPAI H5N1 viruses have spread to over sixty countries on three continents and are endemic among poultry in southeast Asia and Africa. Additionally, H9N2 infections are enzootic among poultry globally and sporadically infect humans, whereas both low and highly pathogenic AIVs of H7 subtype (e.g., H7N2, H7N3 and H7N7) continue to cause sporadic outbreaks. In 2013, a new AIV strain of the H7N9 subtype unexpectedly emerged in China and has since caused more than 1,568 human infections and 616 deaths as of 27 May 2021. Although human-to-human transmission has been limited, AIVs continue to produce variants. The genetic reassortment of the avian and human/porcine influenza viruses or gene mutations can result in virus replication in the upper respiratory tract of humans and generate novel pandemic influenza viruses as happened in the 2009 pandemic.

Antigenic drift in seasonal influenza viruses can substantially limit the duration of immunity conferred by infection or vaccination and is the reason influenza vaccine components are updated every year. The success of seasonal influenza vaccines is mainly dependent on the match between the vaccine constituents and the circulating strains, antigenic distance, attack rate and pre-existing antibodies. Antigenic shift, whether due to genomic reassortment between two or more influenza A viruses or adaptation of avian or swine influenza virus in humans, can lead to successful person-to-person transmission and, ultimately, an influenza pandemic. To address the issue of antigenic drift and antigenic shift in influenza A viruses, a universal influenza vaccine is needed.

### SUMMARY

Immunogenic compositions are provided. In certain embodiments, the immunogenic composition comprises a full-length nucleoprotein (NP) of a H7N9 influenza virus with or without expressing 22 amino acid residues of an Autophagy-Inducing Peptide C5 (AIP-C5) from a CFP10 protein of Mycobacterium tuberculosis, or a functional fragment thereof, and a pharmaceutically acceptable carrier.

In certain embodiments, the immunogenic composition comprises a NP (e.g., full-length or a functional fragment thereof (e.g., epitope)) of a H7N9 influenza virus that also expresses 22 amino acid residues of an AIP-C5 from a CFP10 protein of Mycobacterium tuberculosis. In certain embodiments, the immunogenic composition comprises a NP (e.g., full-length or a functional fragment thereof (e.g., epitope)) of a H7N9 influenza virus that does not express 22 amino acid residues of an AIP-C5 from a CFP10 protein of Mycobacterium tuberculosis. In certain embodiments, the immunogenic composition comprises one or more immunogenic domains (e.g., HA, HA2, M2e, etc.) with or without the expression of AIP-C5 from a CFP10 protein of Mycobacterium tuberculosis.

The immunogenic composition can be cross-protective against two or more subtypes of influenza viruses when administered to a subject (e.g., a mammal). The immunogenic composition can be cross-protective against at least five subtypes of influenza viruses when administered to a subject. In certain embodiments, the composition confers general immunogenicity protection against the subtypes of viruses selected from the group consisting of H1, H3, H5, H7, H9, and influenza B viruses. In certain embodiments, the composition is cross-protective against two or more subtypes of influenza A or B viruses when administered to a subject.

The full-length NP or functional fragment thereof can comprise SEQ ID NO: 1. The AIP-C5 from a CFP10 protein can comprise SEQ ID NO: 3.

The immunogenic composition can optionally further comprise an adjuvant.

In certain embodiments, the immunogenic composition is formulated to be administered intranasally. Alternatively, the immunogenic composition can be formulated to be administered subcutaneously. In certain embodiments, the immunogenic composition is formulated for oral administration. In certain embodiments, the immunogenic composition is formulated as an aerosol spray.

Certain immunogenic compositions hereof comprise SEQ ID NO: 6, 8, 10, 12, or 14; and a pharmaceutically acceptable carrier. Such immunogenic composition can be cross-protective against two or more subtypes of influenza viruses when administered to a subject.

Human or bovine adenoviral (Ad) vectors are also provided. In certain embodiments, the Ad vector is a bovine Ad type 3 (BAd3). In certain embodiments, the Ad is a human Ad vector (HAd). In certain embodiments, the Ad vector comprises a polynucleotide sequence that encodes a full-length NP from H7N9 influenza virus, a functional fragment thereof, and/or one or more other immunogenic domains (e.g., HA, HA2, M2e, etc.) of an influenza virus. The polynucleotide sequence of the Ad can further encode AIP-C5 from the CFP10 protein of Mycobacterium tuberculosis. The AIP-C5 can comprise 22 amino acid residues (e.g., SEQ ID NO: 4). In certain embodiments, the polynucleotide sequence comprises SEQ ID NO: 2. In certain embodiments, the polynucleotide sequence further comprises SEQ ID NO: 4. In certain embodiments, the polynucleotide sequence comprises at least SEQ ID NO: 2 and SEQ ID NO: 4.

In certain embodiments of the Ad vector, the full-length NP, functional fragment thereof, and/or one or more other immunogenic domains of an influenza virus comprises SEQ ID NO: 1. In certain embodiments, the AIP-C5 of the Ad virus comprises SEQ ID NO: 3 (e.g., and encodes SEQ ID NO: 4).

At least the E1 and E3 regions of the Ad vector can be deleted. In certain embodiments where at least the E1 and E3 regions of the Ad vector are deleted, the polynucleotide sequence of the full-length NP, functional fragment thereof and/or one or more other immunogenic domains of an influenza virus is inserted in the deleted E1 region. In certain embodiments of the Ad vector where at least the E1 and E3 regions are deleted, SEQ ID NO: 3 is inserted into the deleted E1 region of the Ad.

The Ad can provide protection against infection by various subtypes of viruses selected from the group consisting of H1, H3, H5, H7, H9, and influenza B viruses.

Human or bovine Ad vectors are also provided that comprise a polynucleotide sequence comprising SEQ ID NO: 5, 7, 9, 11, 13, or 15, or a functional fragment thereof.

Methods of generating a general immunogenicity against a heterosubtypic influenza virus in a subject are also provided. In at least one embodiment, the method comprises administering to a subject an effective amount of an immunogenic composition hereof or an Ad vector hereof. The administration can be intranasal. The administration can be subcutaneous. The administration can be intramuscular. The composition or Ad vector can be administered orally. The composition or Ad vector can be administered as an aerosol spray.

In certain embodiments, the method provides a general immunogenicity protection to the subject against various subtypes of viruses. For example, and without limitation, the various subtypes of viruses can be selected from the group consisting of H1, H3, H5, H7, H9, and influenza B viruses. In certain embodiments, administration of the effective amount of the immunogenic composition or the Ad vector induces a dose-dependent increase in cell-mediated immunity in the subject. The subject can be a mammal. The subject can be a human.

### BRIEF DESCRIPTION OF DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1A is a schematic representation of genomic structures of HAd-ΔE1E3 (HAd5 E1 and E3 deleted empty vector), HAd-NP(H7N9) and HAd-C5-NP(H7N9). The drawings are not to scale. The gene cassette [NP(H7N9) or C5-NP(H7N9)] was under the control of the cytomegalovirus (CMV) promoter and the bovine growth hormone (BGH) polyadenylation signal. (LITR, left inverted terminal repeat; RITR, right ITR; ΔE1, deletion of E1 region; ΔE3, deletion of E3 region; C5, AIP-C5; NP, nucleoprotein)

FIG. 1B is an immunoblot confirming expression of NP(H7N9) or C5-NP(H7N9) in HAd-NP(H7N9)- or HAd-C5-NP(H7N9)-infected 293 cells. Mock or HAd-ΔE1E3 infected cell extracts were used as negative control and the molecular weight marker is shown on the left.

FIGS. 1C and 1D show outlines of one-dose and two-dose animal inoculation studies, respectively.

FIGS. 2A-2D show enzyme-linked immunosorbent assay (ELISA) quantitative data relating to the immunogenicity (i.e., antibody response) in 6-8-week old BALB/c mice (5 animals/group) that were immunized intranasally (i.n.) with a single-dose of HAd-NP(H7N9) or HAd-C5-NP(H7N9), with FIG. 2A showing nucleoprotein (NP)-specific IgG, FIG. 2B showing IgG1, FIG. 2C showing IgG2a, and FIG. 2D showing IgA, all taken from blood samples collected four weeks post-immunization.

FIGS. 2E-2G show ELISA quantiative data relating to mucosal NP-specific antibody levels in the mice of FIGS. 2A-2D taken from lung washes collected four weeks post-immunization, with FIG. 2E showing IgG, FIG. 2F showing IgG1, FIG. 2G showing IgG2a, and FIG. 2H showing IgA. ELISA data in FIGS. 2A-2H are shown as the mean±standard deviation (SD) of the optical density (OD) readings.

FIGS. 2I-2K show data related to the enhancement in the number of NP-specific interferon gamma (IFN-γ) secreting CD8 T cells following immunization with HAd-C5-NP(H7N9) at 4 weeks post-vaccination in the spleens (FIG. 2I), mediastinal lymph node (FIG. 2J), and lung mononuclear (MN) cells (FIG. 2K) by enumerating NP-specific IFN-γ secreting CD8 T cells by ELISpot using the NP-147 peptide. (ns, non-significant at p<0.05; *, significant at p<0.05; **, significant at p<0.01; ***, significant at p<0.001; and ****, significant at p<0.0001). FIG. 2K also shows the symbol legend applicable to FIGS. 2A-2K.

FIGS. 3A-3D show data relating to the protection efficacy of a single-dose regimen of HAd-NP(H7N9) or HAd-C5-NP(H7N9) at 4 weeks post-booster, where immunized animal groups were challenged with 2 lethal doses 50 (LD₅₀) of A/Puerto Rico/8/1934(H1N1) (FIGS. 3A-3B) or 5 LD₅₀ of A/Hong Kong/l/68(H3N2) (FIGS. 3C-3D). FIGS. 3A and 3C show morbidity data, and FIGS. 3B and 3D show mortality data after challenge.

FIGS. 3E, 3F and 3G show data from groups challenged with 100 mouse infectious dose 50 (MID₅₀) of A/chukkar/MN/14951-7/1998 (H5N2) (FIG. 3E), A/goose/Nebraska/17097/2011 (H7N9) (FIG. 3F), or A/Hong Kong/1073/1999 (H9N2) (FIG. 3G) influenza virus where, at 3 days post-challenge, the lungs were collected and lung viral titers were determined. The data are shown as mean Log₁₀ TCID₅₀ or egg infectious dose 50 (EID₅₀), and the detection limit was 0.5 Log₁₀ tissue infectious dose 50 (TCID₅₀) or EID₅₀ per ml. (ns, non-significant at p<0.05; *, significant at p<0.05; **, significant at p<0.01; ***, significant at p<0.001; and ****, significant at p<0.0001). FIG. 3G also shows the symbol legend applicable to FIGS. 3A-3G.

FIGS. 4A-4D show data related to the development of NP-specific IgG (FIG. 4A), IgG1 (FIG. 4B), IgG2a (FIG. 4C), and IgA (FIG. 4D) antibody responses measured by ELISA three weeks post-boost from blood taken from 6-8-week old BALB/c mice (5 animals/group) that were immunized i.n. twice with HAd-NP(H7N9) or HAd-C5NP(H7N9).

FIGS. 4E-4H show the development of mucosal NP-specific IgG (FIG. 4E), IgG1 (FIG. 4F), IgG2a (FIG. 4G), and IgA (FIG. 411) antibody responses measured by ELISA using lung washes collected four weeks post-boost. For FIGS. 4A-4H, ELISA data are shown as the mean±SD of the OD readings.

FIGS. 4I-4K show data related to the enhancement in the number of NP-specific IFN-γ secreting CD8 T cells following immunization with HAd-C5-NP(H7N9), with samples taken at 3 weeks post-booster in the spleen (FIG. 4I), mediastinal lymph node (FIG. 4J), and lung MN Cells (FIG. 4K) by enumerating NP-specific IFN-γ secreting CD8 T cells by ELISpot using the NP-147 peptide. (ns, non-significant at p<0.05; *, significant at p<0.05; **, significant at p<0.01; ***, significant at p<0.001; and ****, significant at p<0.0001). FIG. 4K also shows the symbol legend applicable to FIGS. 4A-4K.

FIGS. 5A and 5C show morbidity data and FIGS. 5B and 5D show mortality data taken at 3 weeks post-booster from immunized animal groups challenged with 2 LD₅₀ of A/Puerto Rico/8/1934(H1N1) (FIGS. 5A and 5B) or 5 LD₅₀ of A/Hong Kong/1/68(H3N2) (FIGS. 5C and 5D).

FIGS. 5E, 5F and 5G show data from lungs collected and lung viral titers determined 3 days post-challenge with 100 MID₅₀ of A/chukkar/MN/14951-7/1998(H5N2) (FIG. 5E), A/goose/Nebraska/17097/2011(H7N9) (FIG. 5F) or A/Hong Kong/1073/1999(H9N2) (FIG. 5G) influenza virus. The data are shown as mean Log₁₀ TCID₅₀ or EID₅₀, and the detection limit was 0.5 Log₁₀ TCID₅₀ or EID₅₀ per ml. (ns, non-significant at p<0.05; *, significant at p<0.05; **, significant at p<0.01; ***, significant at p<0.001; and ****, significant at p<0.0001). FIG. 5G also shows the symbol legend applicable to FIGS. 5A-5G.

FIG. 6 shows histipathology images of lung tissue sections. Mice were mock-immunized (PBS) or immunized intranasally (i.n.) with 10⁸ p.f.u. of HAd-ΔE1E3, HAd-NP(H7N9), or HAd-C5-NP(H7N9). Representative pictures of each group are shown at 1, 4, and 8 days post immunization (H&E, 200X).

FIGS. 7A-7D show graphical data of histopathological scores of the lung tissue sections from the HAd-ΔE1E3-, HAd-NP(H7N9)-, or HAd-C5-NP(H7N9)-immunized mice. Six to 8 week-old BALB/c mice were mock-inoculated (PBS) or inoculated intranasally (i.n.) once with 10⁸ p.f.u./animal of HAd-ΔE1E3, HAd-NP(H7N9), or HAd-C5-NP(H7N9). At 1 (FIG. 7A), 2 (FIG. 7B), 4 (FIG. 7C), and 8 (FIG. 7D) days post immunization, animals were euthanized at and the lung tissue samples were collected and processed for histopathology. The tissue sections were scored by a board-certified veterinary pathologist unaware of the animal groups. FIG. 7D also shows a symbol legend for the symbols in FIGS. 7A-7D.

FIGS. 8A-8C show volcano plots of differential expression (DE) genes in the lungs of mice at 24 hours post-inoculation with HAd-ΔE1E3 (FIG. 8A), HAd-NP(H7N9) (FIG. 8B), or HAd-C5-NP(H7N9) (FIG. 8C) compared to the PBS group using Mouse Autophagy RT² Profiler™ PCR Array (QIAGEN Sciences Inc., Germantown, MD). FIG. 8C also includes a key for the symbols in the plots of FIGS. 8A-8C.

FIG. 9 is a schematic representation of genomic structures of HAd and BAd vectors carrying the full HA gene of H5N1, HA2 (stem domain of H5N1 HA) with or without IgE secretory signal, HA1 signal peptide (SP), and ectodomains of M2 (M2e) of H5N1and H7N9, or NP of H7N9. These gene constructs were expressed with AIP-C5. The drawings are not to scale. Each gene cassette is under the control of the cytomegalovirus (CMV) promoter and the bovine growth hormone (BGH) polyadenylation signal. (LITR, left inverted terminal repeat; RITR, right ITR; ΔE1, deletion of E1 region; ΔE3, deletion of E3 region; C5, AIP-C5; HA, hemagglutinin; NP, nucleoprotein).

FIG. 10A is an immunoblot that confirms expression of HA-C5, IgE-HA2-C5, IgE-HA2-4XM2e-C5, SP-HA2-C5, or SP-HA2-4XM2e-C5 in HAd vectors-infected 293 cells. Mock or HAd-ΔE1E3 infected cell extracts were used as negative controls and the molecular weight marker is shown on the left.

FIG. 10B is an immunoblot that confirms expression of HA-C5, IgE-HA2-C5, IgE-HA2-4XM2e-C5, SP-HA2-C5, or SP-HA2-4XM2e-C5 in BAd vectors-infected BHH-F5 cells. Mock or BAd-ΔE1E3 infected cell extracts were used as negative controls and the molecular weight marker is shown on the left.

While the present disclosure is susceptible to various modifications and alternative forms, exemplary embodiments thereof are shown by way of example in the drawings and are herein described in detail.

### Sequence Listings

The sequences herein (SEQ ID NOS: 1-15) are also provided in computer readable form encoded in a file filed herewith and incorporated herein by reference. The information recorded in computer readable form is identical to the written Sequence Listing provided below, pursuant to 37 C.F.R. § 1.821(f).

SEQ ID NO: 1 is the nucleoprotein (NP) of influenza virus H7N9
has an amino acid sequence of:
MASQGTKRSYEQMETGGERQNATEIRASVGRMVSGIGRFYIQMCTELKLSDNEGRLIQNSI
TIERMVLSAFDERRNRYLEEHPSAGKDPKKTGGPIYRRRDGKWVRELILYDKEEIRRIWRQ
ANNGEDATAGLTHLMIWHSNLNDATYQRTRALVRTGMDPRMCSLMQGSTLPRRSGAAGA
AVKGIGTMVMELIRMIKRGINDRNFWRGENGRRTRIAYERMCNILKGKFQTAAQRAMMDQ
VRESRNPGNAEIEDLIFLARSALILRGSVAHKSCLPACVYGLAVASGYDFEREGYSLVGIDPF
RLLQNSQVFSLIRPNENPAHKSQLVWMACHSAAFEDLRVSSFIRGTRMVPRGQLSTRGVQIA
SNENMEAMDSNTLELRSRYWAIRTRSGGNTNQQRASAGQVSVQPTFSVQRNLPFERATIMA
AFTGNTEGRTSDMRTEIIRMMESARPEDVSFQGRGVFELSDEKATNPIVPSFDMNNEGSYFF
GDNAEEYDN.
SEQ ID NO: 2 is the DNA sequence that encodes SEQ ID NO: 1:
ATGGCTTCCCAGGGCACAAAGAGGTCTTACGAGCAGATGGAGACCGGCGGAGAGAGAC
AGAACGCCACAGAGATCAGAGCTAGCGTGGGACGGATGGTGTCCGGAATCGGCCGCTT
CTACATCCAGATGTGCACCGAGCTGAAGCTGTCCGACAACGAGGGCCGGCTGATCCAG
AACTCCATCACAATCGAGCGCATGGTGCTGTCTGCCTTTGACGAGAGGAGAAACAGATA
CCTGGAGGAGCACCCTTCTGCTGGAAAGGATCCAAAGAAGACCGGAGGACCAATCTAC
CGGCGCAGGGACGGCAAGTGGGTGAGAGAGCTGATCCTGTACGATAAGGAGGAGATCA
GACGGATCTGGCGGCAGGCCAACAACGGAGAGGACGCCACCGCTGGCCTGACACACCT
GATGATCTGGCACAGCAACCTGAACGACGCCACCTACCAGCGCACAAGGGCTCTGGTG
AGGACCGGAATGGATCCCAGAATGTGCTCCCTGATGCAGGGCTCTACACTGCCTCGCAG
GTCCGGAGCTGCTGGAGCTGCTGTGAAGGGAATCGGCACCATGGTCATGGAGCTGATCA
GAATGATCAAGCGGGGCATCAACGATCGCAACTTCTGGAGGGGAGAGAACGGCAGACG
GACCCGCATCGCCTACGAGAGGATGTGCAACATCCTGAAGGGCAAGTTTCAGACAGCC
GCTCAGAGGGCCATGATGGACCAGGTGAGAGAGTCCCGGAACCCCGGAAACGCTGAGA
TCGAGGATCTGATCTTCCTGGCTCGGTCTGCTCTGATCCTGAGGGGAAGCGTGGCTCAC
AAGTCCTGCCTGCCAGCTTGCGTGTACGGACTGGCCGTGGCTTCTGGCTACGACTTTGA
GCGGGAGGGATACAGCCTGGTGGGCATCGATCCCTTCCGCCTGCTGCAGAACTCTCAGG
TGTTTAGCCTGATCAGGCCAAACGAGAACCCCGCCCACAAGAGCCAGCTGGTGTGGAT
GGCTTGTCACTCCGCCGCTTTCGAGGACCTGCGGGTGAGCTCCTTTATCCGCGGCACCA
GGATGGTGCCTAGGGGACAGCTGAGCACAAGAGGCGTGCAGATCGCCTCCAACGAGAA
CATGGAGGCTATGGATTCTAACACCCTGGAGCTGAGAAGCCGGTACTGGGCTATCAGGA
CCAGGAGCGGCGGAAACACAAACCAGCAGAGGGCTTCTGCTGGACAGGTGAGCGTGC
AGCCTACCTTCTCCGTGCAGCGGAACCTGCCATTTGAGCGCGCCACAATCATGGCCGCT
TTCACCGGAAACACAGAGGGCAGAACCTCTGACATGCGGACAGAGATCATCCGCATGAT
GGAGTCCGCCAGGCCAGAGGACGTGAGCTTCCAGGGAAGAGGCGTGTTTGAGCTGAGC
GACGAGAAGGCTACAAACCCCATCGTGCCTTCTTTCGATATGAACAACGAGGGAAGCTA
CTTCTTTGGCGACAACGCCGAGGAGTACGATAACTGA.

SEQ ID NO: 3 is an amino acid sequence for an Autophagy-Inducing Peptide C5 (AIP-C5) from the CFP10 protein of Mycobacterium tuberculosis.

MAAQAAVVRFQEAANKQKQELD.

SEQ ID NO: 4 is the DNA sequence that encodes SEQ ID NO: 3:

ATGGCCGCTCAGGCCGCTGTGGTGAGATTCCAGGAGGCCGCTAACAAGCA
GAAGCAGGAGCTGGAC.

SEQ ID NO: 5 is an amino acid sequence for NP147 peptide (H-2K^{d}-restricted CTL epitope for NP): TYQRTRALV.

SEQ ID NO: 6 is an amino acid sequence for the full-length H5N1 HA (signal peptide (amino acids 1-16), HA1 (amino acids 17-346) and -HA2 (amino acids 347-568)- P2A (amino acids 574-592)-C5 (amino acids 593-613):

MEKIVLLFAIVSLVKSDQICIGYHANNSTEQVDTIMEKNVTVTHAQDILEKKHNGKLCDLD
GVKPLILRDCSVAGWLLGNPMCDEFINVPEWSYIVEKANPVNDLCYPGDENDYEELKHLLS
RINHFEKIQIIPKSSWSSHEASLGVSSACPYQGKSSFFRNVVWLIKKNSTYPTIKRSYNNTNQ
EDLLVLWGIHHPNDAAEQTKLYQNPTTYISVGTSTLNQRLVPRIATRSKVNGQSGRMEFFW
TILKPNDAINFESNGNFIAPEYAYKIVKKGDSTIMKSELEYGNCNTKCQTPMGAINSSMPFH
NIHPLTIGECPKYVKSNRLVLATGLRNSPQRERRRKKRGLFGAIAGFIEGGWQGMVDGWY
GYHHSNEQGSGYAADKESTQKAIDGVTNKVNSIIDKMNTQFEAVGREFNNLERRIENLNK
KMEDGFLDVWTYNAELLVLMENERTLDFHDSNVKNLYDKVRLQLRDNAKELGNGCFEF
YHKCDNECMESVRNGTYDYPQYSEEARLKREEISGVKLESIGIYQILSIYSTVASSLALAIMV
AGLSLWMCSNGSLQCRICIGPGPGATNFSLLKQAGDVEENPGPAAQAAVVRFQEAANKQK
QELD*
SEQ ID NO: 7 is the DNA sequence that encodes SEQ ID NO: 6:
ATGGAGAAAATAGTACTTCTCTTTGCTATCGTTAGCCTGGTCAAGTCTGACCAGATCTG
CATCGGCTACCATGCTAACAACAGTACTGAGCAAGTTGATACTATTATGGAGAAGAAT
GTTACTGTCACACATGCTCAGGACATCCTGGAAAAGAAGCACAACGGGAAGCTGTGCG
ATCTGGATGGTGTGAAGCCTCTCATTCTTAGAGACTGCTCAGTGGCCGGTTGGCTCCTA
GGCAACCCTATGTGTGATGAGTTCATCAACGTTCCGGAGTGGAGCTATATCGTGGAAA
AAGCTAATCCAGTAAATGACCTTTGCTACCCTGGTGACTTTAACGATTATGAAGAACTA
AAACACCTGCTGAGTCGGATTAACCACTTTGAGAAAATCCAGATTATCCCTAAGAGCA
GCTGGTCATCTCATGAGGCCAGCCTGGGAGTCTCCTCAGCCTGTCCATACCAAGGGAA
ATCTTCCTTCTTCCGGAATGTGGTCTGGCTGATCAAAAAAAATTCAACGTACCCCACAA
TAAAGAGATCCTACAACAATACTAATCAAGAAGACTTACTCGTGCTTTGGGGAATTCA
CCATCCCAACGATGCCGCTGAGCAGACCAAACTGTATCAGAACCCCACCACTTACATC
AGCGTGGGCACCAGCACCTTGAACCAGCGCCTTGTCCCGCGCATTGCCACAAGGTCTA
AGGTAAACGGACAGTCAGGCAGGATGGAATTTTTCTGGACAATACTGAAGCCAAATGA
TGCCATAAACTTTGAATCCAATGGCAATTTTATCGCTCCAGAGTATGCCTACAAAATAG
TAAAAAAAGGAGACTCAACTATAATGAAGTCAGAACTAGAATATGGAAACTGCAACA
CCAAGTGCCAGACACCCATGGGTGCTATCAATTCGTCCATGCCTTTCCATAACATCCAC
CCACTGACGATTGGGGAGTGTCCCAAGTATGTGAAGAGTAACCGGTTGGTGCTCGCCA
CCGGACTTCGAAATTCCCCTCAGAGAGAACGTCGTCGGAAGAAACGCGGGCTGTTTGG
AGCAATCGCTGGCTTCATTGAAGGGGGATGGCAGGGTATGGTGGATGGCTGGTACGGC
TATCACCACAGCAACGAACAGGGCTCTGGATACGCAGCAGACAAGGAGTCTACCCAGA
AGGCGATTGATGGGGTCACCAATAAAGTCAACTCGATCATTGACAAAATGAATACACA
GTTTGAAGCAGTAGGTCGCGAGTTTAATAATTTGGAAAGGCGAATTGAGAATCTTAAC
AAGAAGATGGAGGATGGCTTTTTGGATGTGTGGACCTACAATGCGGAGCTCCTCGTGC
TGATGGAAAATGAGCGAACGCTGGACTTCCATGACTCTAACGTGAAAAACCTCTACGA
TAAGGTTCGGCTCCAACTCAGAGACAATGCGAAGGAACTGGGGAACGGCTGCTTCGAG
TTCTACCACAAGTGTGACAATGAATGCATGGAGAGCGTCAGAAATGGCACATATGACT
ATCCACAGTACAGCGAGGAGGCAAGATTGAAGAGGGAGGAAATTTCTGGGGTTAAGC
TGGAGTCCATTGGAATCTACCAGATCTTAAGTATCTATAGTACTGTGGCCAGTTCTCTG
GCCTTGGCTATCATGGTCGCAGGGTTGTCGCTATGGATGTGCAGCAACGGCTCGCTGCA
GTGTAGGATTTGTATTGGTCCTGGGCCCGGAGCTACAAACTTCAGCCTCCTGAAGCAGG
CCGGCGACGTGGAGGAGAACCCCGGGCCAGCAGCCCAGGCAGCTGTGGTGAGGTTCC
AGGAGGCCGCCAACAAACAAAAACAAGAGCTGGATTGA.
SEQ ID NO: 8 is an amino acid sequence for a secretory signal
(IgE) (amino acids 1-19)-HA1Ahead(cys52-277) (amino acids
20-61 and 66-122)-HA2ATMDACD (H5N1) (amino acids 123-308)-P2A
(amino acids 314-332)-C5 (amino acids 333-353):
MKLPVRLLVLMFWIPASSSDQICIGYHANNSTEQVDTIMEKNVTVTHAQDILEKKHNGKLC
GGGGCNTKCQTPMGAINSSMPFHNIHPLTIGECPKYVKSNRLVLATGLRNSPQRERRRKKR
GLFGAIAGFIEGGWQGMVDGWYGYHHSNEQGSGYAADKESTQKAIDGVTNKVNSIIDKM
NTQFEAVGREFNNLERRIENLNKKMEDGFLDVWTYNAELLVLMENERTLDFHDSNVKNLY
DKVRLQLRDNAKELGNGCFEFYHKCDNECMESVRNGTYDYPQYSEEARLKREEISGVKLE
SIGIYQIGPGPGATNFSLLKQAGDVEENPGPAAQAAVVRFQEAANKQKQELD*
SEQ ID NO: 9 is the DNA sequence that encodes SEQ ID NO: 8:
ATGAAACTGCCAGTCCGACTCCTTGTTTTGATGTTCTGGATACCTGCCAGCTCTTCAGA
CCAGATCTGCATTGGCTACCATGCGAACAATTCTACAGAGCAGGTGGACACCATCATG
GAAAAGAATGTAACTGTGACACATGCTCAAGACATCCTGGAGAAGAAGCACAATGGG
AAACTCTGTGGGGGAGGTGGGTGTAATACCAAGTGCCAAACGCCCATGGGGGCCATCA
ACAGTTCGATGCCCTTCCACAACATCCATCCCCTGACAATAGGAGAGTGTCCCAAGTAT
GTGAAATCCAACAGATTGGTTCTGGCTACTGGCTTGCGGAACAGCCCTCAAAGAGAAA
GACGCCGGAAAAAGAGGGGCCTTTTTGGGGCCATTGCTGGCTTCATTGAAGGTGGCTG
GCAGGGCATGGTGGATGGATGGTACGGCTATCACCACTCCAATGAACAAGGTTCTGGT
TATGCTGCTGATAAAGAATCCACACAGAAGGCCATTGATGGAGTCACTAACAAAGTCA
ACAGCATTATCGATAAGATGAACACCCAGTTTGAGGCAGTGGGCCGGGAATTCAACAA
CCTGGAACGACGTATTGAAAACTTAAACAAGAAAATGGAGGATGGGTTTCTGGATGTG
TGGACATACAACGCAGAGCTCCTAGTGCTTATGGAAAATGAGAGGACTCTGGACTTTC
ATGATTCAAATGTTAAAAATCTTTATGACAAAGTCCGCCTACAGCTCAGGGACAATGC
CAAGGAGCTGGGGAATGGCTGCTTTGAGTTCTACCACAAGTGTGACAACGAGTGCATG
GAGAGTGTCAGAAATGGAACCTATGATTACCCACAGTACAGTGAAGAGGCCAGGCTGA
AGCGGGAGGAAATCTCTGGAGTGAAGCTGGAGTCCATCGGTATATACCAGATTGGACC
GGGACCTGGAGCGACCAACTTCAGCCTGTTGAAGCAAGCAGGGGACGTAGAGGAGAA
CCCTGGCCCAGCAGCCCAGGCTGCTGTGGTTCGCTTCCAGGAAGCAGCCAATAAGCAG
AAACAGGAATTAGACTGA.

SEQ ID NO: 10 is an amino acid sequence for a secretory signal (IgE) (amino acids 1-19)-HA1Δhead(cys52-277) (amino acids 20-61 and 66-122)-HA2ATMDACD (H5N1) (amino acids 123-308)- linker (amino acids 309-313)- M2e(H5N1) (amino acids 314-336)-M2e(H5N1) (amino acids 370-392)-M2e(H7N9) (amino acids 398-420)-P2A (amino acids 426-444)-C5 (amino acids 445-465):

MKLPVRLLVLMFWIPASSSDQICIGYHANNSTEQVDTIMEKNVTVTHAQDILEKKHNGKLC
GGGGCNTKCQTPMGAINSSMPFHNIHPLTIGECPKYVKSNRLVLATGLRNSPQRERRRKKR
GLFGAIAGFIEGGWQGMVDGWYGYHHSNEQGSGYAADKESTQKAIDGVTNKVNSIIDKM
NTQFEAVGREFNNLERRIENLNKKMEDGFLDVWTYNAELLVLMENERTLDFHDSNVKNL
YDKVRLQLRDNAKELGNGCFEFYHKCDNECMESVRNGTYDYPQYSEEARLKREEISGVKL
ESIGIYQIGPGPGSLLTEVETPTRNEWECRCSDSSDGPGPGSLLTEVETPTRTGWECNCSGSS
EGPGPGSLLTEVETPTRNEWECRCSDSSDGPGPGSLLTEVETPTRTGWECNCSGSSEGPGPG
ATNFSLLKQAGDVEENPGPAAQAAVVRFQEAANKQKQELD*.
SEQ ID NO: 11 is the DNA sequence that encodes SEQ ID NO: 10:
ATGAAGCTACCAGTCAGACTATTGGTGCTGATGTTCTGGATTCCTGCGAGCAGTTCTGA
CCAGATCTGTATCGGCTACCATGCAAACAACAGCACAGAGCAGGTTGATACCATCATG
GAGAAGAACGTCACAGTGACACATGCCCAGGACATCCTGGAGAAGAAGCACAATGGA
AAACTGTGTGGTGGTGGGGGATGCAACACAAAGTGCCAGACCCCCATGGGAGCGATTA
ATTCCTCCATGCCTTTTCACAACATCCATCCTCTCACCATTGGTGAATGTCCCAAATATG
TTAAATCGAATAGGCTCGTACTGGCCACAGGGTTAAGGAATTCACCACAGCGGGAGAG
ACGGAGGAAGAAGAGGGGACTCTTTGGGGCAATTGCTGGCTTCATCGAAGGCGGCTGG
CAGGGCATGGTGGATGGATGGTATGGATACCACCACAGTAACGAGCAAGGAAGCGGC
TATGCTGCTGACAAAGAAAGCACCCAGAAAGCCATTGATGGAGTCACCAACAAGGTGA
ATTCTATAATAGACAAGATGAACACACAGTTTGAGGCAGTTGGTCGGGAGTTTAATAA
CCTGGAGCGCCGCATTGAGAATCTGAATAAAAAAATGGAGGACGGATTCCTGGATGTC
TGGACCTACAACGCAGAGTTGCTTGTTCTCATGGAAAATGAGCGGACCCTGGACTTTCA
TGACTCTAATGTGAAGAACCTGTATGATAAAGTGAGGCTGCAATTGAGAGATAATGCT
AAGGAGCTTGGAAATGGCTGCTTTGAATTCTACCACAAGTGTGATAATGAGTGCATGG
AATCTGTGCGAAACGGCACCTATGACTACCCGCAGTACTCCGAAGAAGCCCGTCTGAA
GCGAGAAGAAATCAGTGGTGTCAAACTGGAGAGCATAGGGATCTACCAGATTGGCCCC
GGGCCGGGATCTCTCCTTACTGAAGTAGAGACCCCAACCAGAAATGAATGGGAGTGCC
GCTGCAGCGATTCGTCAGACGGCCCAGGTCCCGGGTCCTTGCTGACAGAAGTTGAAAC
ACCCACGCGAACGGGTTGGGAATGTAACTGTTCCGGGTCCTCAGAAGGGCCTGGGCCT
GGTTCTCTACTGACTGAGGTGGAAACTCCCACTCGTAACGAGTGGGAATGCAGATGTA
GTGACTCCAGCGACGGGCCAGGTCCAGGATCACTCTTGACAGAGGTAGAGACTCCCAC
TAGAACTGGCTGGGAGTGTAACTGCAGTGGCAGTTCTGAAGGGCCAGGCCCTGGCGCC
ACGAACTTCTCCCTTTTAAAGCAGGCCGGAGATGTGGAGGAGAACCCAGGGCCTGCTG
CTCAAGCAGCTGTGGTCAGGTTCCAGGAGGCCGCCAACAAACAGAAGCAAGAGCTGG
ATTGA.

SEQ ID NO: 12 is an amino acid sequence for a signal peptide (HA/H5N1) (amino acids 1-12)- HA1Δhead(cys52-277) (amino acids 13-58 and 63-119)-HA2 (H5N1) (amino acids 120-341)-P2A (amino acids 347-365)-C5 (amino acids 366-382):

MEKIVLLFAIVSLVKSDQICIGYHANNSTEQVDTIMEKNVTVTHAQDILEKKHNGKLCGGG
GCNTKCQTPMGAINSSMPFHNIHPLTIGECPKYVKSNRLVLATGLRNSPQRERRRKKRGLF
GAIAGFIEGGWQGMVDGWYGYHHSNEQGSGYAADKESTQKAIDGVTNKVNSIIDKMNTQ
FEAVGREFNNLERRIENLNKKMEDGFLDVWTYNAELLVLMENERTLDFHDSNVKNLYDK
VRLQLRDNAKELGNGCFEFYHKCDNECMESVRNGTYDYPQYSEEARLKREEISGVKLESIG
IYQILSIYSTVASSLALAIMVAGLSLWMCSNGSLQCRICIGPGPGATNFSLLKQAGDVEENPG
PAAQAAVVRFQEAANKQK*
SEQ ID NO: 13 is the DNA sequence that encodes SEQ ID NO: 12:
ATGGAGAAAATTGTGCTACTGTTTGCTATAGTCAGTCTGGTGAAATCTGACCAGATCTG
TATTGGCTACCATGCCAACAACTCCACAGAACAGGTCGACACTATAATGGAAAAGAAT
GTTACGGTGACACACGCCCAGGACATCTTGGAGAAGAAGCACAATGGGAAGTTGTGTG
GGGGAGGTGGATGCAACACCAAGTGCCAGACCCCCATGGGCGCCATCAATTCATCCAT
GCCCTTCCACAACATACACCCTCTCACCATCGGAGAATGTCCAAAATATGTGAAGTCCA
ACCGTCTCGTCTTGGCAACTGGTCTTCGGAACAGTCCACAGAGAGAACGCCGCCGCAA
AAAGAGAGGTCTTTTTGGAGCTATTGCTGGTTTCATCGAAGGCGGCTGGCAGGGCATG
GTTGATGGATGGTACGGGTATCATCATAGCAACGAGCAAGGCTCTGGATATGCAGCAG
ACAAAGAATCTACTCAGAAGGCCATTGATGGTGTCACCAACAAGGTGAACAGCATCAT
AGACAAGATGAACACACAGTTTGAAGCTGTGGGCCGGGAGTTTAACAATCTCGAACGA
AGGATTGAGAACCTGAATAAGAAAATGGAAGATGGGTTCCTGGATGTCTGGACATACA
ACGCTGAGCTCCTTGTTCTGATGGAGAATGAGCGGACGCTGGACTTCCATGACTCGAAT
GTTAAAAATTTGTATGATAAAGTAAGGCTGCAGCTGAGGGACAATGCCAAGGAGCTAG
GCAACGGCTGCTTTGAGTTCTACCACAAGTGCGATAATGAATGCATGGAGTCCGTCAG
AAATGGAACCTATGACTACCCTCAATATAGTGAAGAGGCCCGATTAAAAAGGGAGGA
GATCTCCGGCGTAAAGCTGGAGAGCATTGGAATTTACCAGATTCTTTCAATCTACAGTA
CTGTGGCCTCTTCTCTGGCCCTGGCTATCATGGTGGCTGGGCTAAGTCTCTGGATGTGT
AGCAATGGTAGCTTACAGTGTAGAATCTGCATCGGGCCCGGGCCTGGCGCGACAAACT
TCTCATTGCTGAAGCAGGCTGGAGATGTGGAGGAAAACCCAGGGCCGGCCGCGCAAGC
AGCAGTGGTACGTTTCCAGGAAGCAGCCAATAAACAAAAATGA.

SEQ ID NO: 14 is an amino acid sequence for a signal peptide (HA/H5N1) (amino acids 1-16)- HA1Δhead(cys52-277) (amino acids 17-58 and 63-119)-HA2 (H5N1) (amino acids 120-341)-P2A (amino acids 347-365)-M2e(H5N1) (amino acids 366-388)-M2e(H7N9) (amino acids 394-416)-M2e(H5N1) (amino acids 422-444)-M2e(H7N9) (amino acids 450-472)-P2A (amino acids 478-496)-C5 (amino acids 497-517):

MEKIVLLFAIVSLVKSDQICIGYHANNSTEQVDTIMEKNVTVTHAQDILEKKHNGKLCGGG
GCNTKCQTPMGAINSSMPFHNIHPLTIGECPKYVKSNRLVLATGLRNSPQRERRRKKRGLF
GAIAGFIEGGWQGMVDGWYGYHHSNEQGSGYAADKESTQKAIDGVTNKVNSIIDKMNTQ
FEAVGREFNNLERRIENLNKKMEDGFLDVWTYNAELLVLMENERTLDFHDSNVKNLYDK
VRLQLRDNAKELGNGCFEFYHKCDNECMESVRNGTYDYPQYSEEARLKREEISGVKLESIG
IYQILSIYSTVASSLALAIMVAGLSLWMCSNGSLQCRICIGPGPGATNFSLLKQAGDVEENPG
PSLLTEVETPTRNEWECRCSDSSDGPGPGSLLTEVETPTRTGWECNCSGSSEGPGPGSLLTE
VETPTRNEWECRCSDSSDGPGPGSLLTEVETPTRTGWECNCSGSSEGPGPGATNFSLLKQA
GDVEENPGPAAQAAVVRFQEAANKQKQELD*.
SEQ ID NO: 15 is the DNA sequence that encodes SEQ ID NO: 14:
ATGGAGAAGATCGTACTGCTTTTTGCCATAGTTAGCTTGGTCAAGTCTGACCAGATCTG
CATCGGCTACCATGCAAACAATAGCACCGAGCAAGTGGACACCATAATGGAGAAAAA
TGTGACTGTGACACATGCCCAGGACATCTTAGAAAAGAAACACAATGGGAAGCTCTGT
GGGGGAGGGGGCTGTAATACCAAGTGCCAGACCCCCATGGGCGCCATCAACAGCTCCA
TGCCTTTCCACAACATTCACCCCCTGACAATTGGGGAGTGTCCCAAATATGTGAAAAGC
AACAGACTGGTCCTTGCGACTGGCCTACGTAACTCTCCACAACGGGAGCGGAGAAGGA
AAAAGAGAGGTCTTTTTGGAGCAATTGCCGGATTCATTGAAGGCGGCTGGCAGGGAAT
GGTGGATGGATGGTACGGTTATCATCACTCTAACGAGCAGGGCTCCGGGTATGCAGCT
GACAAGGAGTCCACTCAAAAGGCCATTGATGGTGTTACCAACAAAGTGAATTCAATAA
TTGACAAAATGAACACTCAGTTTGAGGCTGTTGGTCGCGAGTTCAACAACCTGGAGCG
TAGAATAGAGAATCTGAACAAGAAGATGGAGGACGGCTTCCTGGACGTGTGGACCTAC
AACGCTGAGTTGCTCGTCCTGATGGAAAACGAAAGGACCTTGGACTTTCATGACAGCA
ATGTAAAGAACCTCTATGATAAGGTCCGGCTCCAGCTGCGGGATAATGCCAAAGAGCT
GGGCAACGGCTGCTTTGAATTCTACCACAAGTGTGACAATGAATGCATGGAAAGTGTT
CGCAATGGCACTTATGACTACCCACAGTACAGTGAAGAAGCTCGACTTAAAAGAGAAG
AGATCAGCGGAGTGAAGCTGGAAAGCATTGGAATCTACCAGATCCTATCCATCTATTC
CACAGTCGCCTCTTCACTAGCTTTGGCCATCATGGTAGCAGGTCTGAGCCTCTGGATGT
GCTCGAATGGAAGTCTGCAGTGCAGGATCTGTATTGGACCAGGACCGGGTGCAACAAA
CTTCTCCCTCCTGAAGCAGGCCGGCGATGTCGAGGAGAACCCGGGGCCTTCGCTGCTC
ACAGAAGTGGAAACTCCTACACGCAACGAGTGGGAATGTCGATGCTCAGATTCTAGTG
ACGGTCCCGGACCAGGCAGCCTTCTGACGGAAGTAGAGACACCAACAAGGACTGGTTG
GGAATGTAACTGTTCAGGCAGCAGCGAGGGGCCTGGACCTGGTTCTTTGCTGACGGAG
GTGGAGACACCTACCAGGAATGAATGGGAGTGCCGCTGTTCTGATTCTTCAGATGGGC
CAGGCCCGGGTTCATTACTCACCGAGGTTGAAACGCCCACCCGGACAGGCTGGGAGTG
CAACTGCAGTGGCAGCAGCGAAGGGCCAGGGCCCGGGGCCACCAATTTTTCCTTACTG
AAACAAGCTGGAGATGTGGAGGAAAATCCTGGGCCCGCAGCACAGGCTGCTGTGGTCA
GATTCCAGGAGGCCGCGAATAAACAGAAGCAAGAATTAGATTGA.

The content of the XML file of the sequence listing (named “PRF69569-02SeqListingXML_16sep.2024.xml” which is 39,888,404 bytes in size, created Sep. 16, 2024, and electronically submitted via Patent Center on Sep. 16, 2024) is incorporated herein by reference in its entirety. The information recorded in computer readable form is identical to the written Sequence Listing provided herein (on paper) pursuant to 37 C.F.R. § 1.821(f).

### DETAILED DESCRIPTION

While the concepts of the present disclosure are illustrated and described in detail in the description herein, results in the description are to be considered as exemplary and not restrictive in character; it being understood that only the illustrative embodiments are shown and described, and that all changes and modifications that come within the spirit of the disclosure are desired to be protected.

The present disclosure generally relates to a universal influenza vaccine. More specifically, the present disclosure relates to methods and compositions of matter for an effective general vaccination against heterosubtypic influenza viruses using an adenoviral vector with E1 and E3 regions removed and expressing the nucleoprotein (NP) of influenza virus H7N9 with or without the presence of the Autophagy-Inducing Peptide C5 (AIP-C5) from the CFP10 protein of Mycobacterium tuberculosis. In certain embodiments, compositions comprise one or more immunogenic domains (e.g., HA, HA2, M2e, etc.) with or without the expression of AIP-C5 from a CFP10 protein of Mycobacterium tuberculosis. Pharmaceutical compositions and methods of use thereof are also provided.

While candidate vaccines can be made for individual influenza strains, it is impractical to prepare significant vaccine stocks for each of the potential pandemic viruses. Moreover, the nature of the pandemic influenza virus is typically known at the time of the pandemic (not beforehand). Therefore, a universal influenza vaccine that can confer adequate protection against seasonal influenza A viruses (H1N1 and H3N2) as well as potential pandemic avian influenza A viruses (H5N1, H7N7, H7N9, and H9N2), and/or influenza B viruses is desirable.

Adenoviral (Ad) vector-based vaccines have demonstrated excellent promise for developing effective vaccines against several pathogens, including the Ebola virus and severe acute respiratory syndrome coronavirus 2 (SARS-CoV-2) in preclinical and clinical studies and have been licensed under Emergency Use Authorization (EUA). Moreover, Ad vector-based influenza vaccines expressing hemagglutinin (HA) (i.e. the principal viral protein that is responsible for binding to host cell receptors), neuraminidase (NA), nucleoprotein (NP), matrix protein 1 (M1), matrix protein 2 (M2), or immunogenic domains or epitopes have shown potential in providing significant protection against influenza viruses in experimental animals or human clinical trials. In addition, Ad vector immunity can be addressed either by using less prevalent HAds or nonhuman Ads as vaccine platforms. Indeed, a nanoparticle-based vaccine carrying the four HAs of seasonal influenza viruses resulted in antibody responses with similar or higher levels than the quadrivalent influenza vaccines in animal models. (see Boyoglu-Barnum et al., Quadrivalent influenza nanoparticle vaccines induce broad protection, Nature 592: 623-628, doi:10.1038/s41586-021-03365-x (2021)). Immunized animals were protected from heterologous viruses due to the development of broadly protective antibody responses to the conserved HA stem region. In a phase I trial, a chimeric HA-based vaccine in healthy adults generated broad and durable cross-reactive antibodies against the HA stalk domain. (see Nachbagauer et al., A chimeric hemagglutinin-based universal influenza virus vaccine approach induces broad and long-lasting immunity in a randomized, placebo-controlled phase I trial, Nat Med 27: 106-114, doi:10.1038/s41591-020-1118-7 (2021)).

The influenza virus internal protein, namely NP, is relatively conserved across multiple subtypes of the influenza virus and serves as a robust inducer of heterosubtypic CD8+ cytotoxic T lymphocyte (CTL) responses following infection. CTL immunity can aid in viral clearance and non-neutralizing antibody responses, which can participate in antibody-dependent cell-mediated cytotoxicity (ADCC) or complement-dependent lysis (CDL) or induction of CD4 T helper cells. It has previously been demonstrated that intramuscular (i.m.) vaccination of mice with human Ad type 5 (HAd5) vector expressing NP of H5N1 virus resulted in approximately 2.4, 1.9, 2.3, 2.4, or 1.4, logs reduction of lung virus titers of H1, H3, H5, H7, and H9 influenza viruses, respectively. (see Hassan et al., Adenovirus vector-based multi-epitope vaccine provides partial protection against H5, H7, and H9 avian influenza viruses, PloS One 12: e0186244, doi:10.1371/journal.pone.0186244 (2017); and Vemula et al., Broadly protective adenovirus-based multivalent vaccines against highly pathogenic avian influenza viruses for pandemic preparedness, PLoS One 8: e62496, doi:10.1371/journal.pone.0062496 (2013)). Some studies have described broad, but partial, protection with Ad or other viral vector-based NP vaccines. (see Vemula (2013), supra; Roy et al., Partial protection against H5N1 influenza in mice with a single dose of a chimpanzee adenovirus vector expressing nucleoprotein, Vaccine 25: 6845-6851, doi:10.1016/j.vaccine.2007.07.035 (2007); and Li et al., Single-dose vaccination of a recombinant parainfluenza virus 5 expressing NP from H5N1 virus provides broad immunity against influenza A viruses, J Virol 87: 5985-5993, doi:10.1128/jvi.00120-13 (2013)). Other conserved influenza antigens like M1, HA2 (HA stalk domain), M1 and/or M2 ectodomain with NP in Ad vectors or other viral vectors have been utilized to broaden further vaccine protection efficacy. (Asthagiri et al., Vaccination with viral vectors expressing NP, M1 and chimeric hemagglutinin induces broad protection against influenza virus challenge in mice, Vaccine 37: 5567-5577, doi: doi.org/10.1016/j.vaccine.2019.07.095 (2019); McMahon et al., Vaccination with viral vectors expressing chimeric hemagglutinin, NP and M1 antigens protects ferrets against influenza virus challenge, Frontiers in Immun 10: 2005, doi:10.3389/fimmu.2019.02005 (2019); Zhou et al., A universal influenza A vaccine based on adenovirus expressing matrix-2 ectodomain and nucleoprotein protects mice from lethal challenge, Mol Ther 18: doi:10.1038/mt.2010.202 (2010); and Wang et al., Improving cross-protection against influenza virus using recombinant vaccinia vaccine expressing NP and M2 ectodomain tandem repeats, Virologica Sinica 34: 583-591, doi:10.1007/s12250-019-00138-9 (2019)). These conventional studies used the systemic route of inoculation to deliver viral vector-based vaccine formulations and led to variable protection efficacy.

The present antigens, compositions, Ad vectors, and methods leverage NP as a target for a universal influenza vaccine. To further enhance T cell immunity of NP, a 22-amino acid long Autophagy-Inducing Peptide (AIP) C5 (AIP-C5) from the secreted CFP10 protein of Mycobacterium tuberculosis (Mtb) can further be incorporated into the compositions hereof (e.g., vaccines). Here, the inclusion of the C5-AIP with the H7N9 NP gene can significantly enhance T cell immune responses and broaden the protective efficacy of an Ad vector-based universal influenza vaccine hereof. For example, intranasal (i.n.) immunization of mice with HAd vector expressing NP(H7N9) or C5-NP(H7N9) conferred complete protection against H1N1, H3N2, H5N2, H7N9, and H9N2 influenza viruses, signifying the importance of the route of immunization (intranasal, i.n.), delivery vector (Ad), influenza antigen (NP), and the AIP-C5 in developing a universal influenza vaccine.

In certain embodiments, a vaccine production system is provided that comprises an Ad. The Ad can comprise an immunogenic composition (e.g., a vaccine) that confers general immunogenicity protection against various subtypes of viruses. As used herein, conferring “immunogenicity protection” means eliciting a protective immune response (e.g., cellular or humoral) in a subject. In certain embodiments, the immunogenic composition (e.g., a vaccine) produced by the Ad can confer, when administered to a subject (e.g., intranasally) general immunogenicity protection against various subtypes of viruses selected from the group consisting of H1, H3, H5, H7, and H9 influenza viruses and/or B influenza viruses.

Human Ads (HAds) are well-known in the art and can be constructed to include one or more of the components described herein. Alternatively, nonhuman Ads, such as chimpanzee, simian, ovine, avian, murine, porcine or bovine Ad vectors (for example, ChAd, Sad, OAd, AAd, Mad, PAd, or BAd vectors) can be used. Typically, the Ad is a replication defective Ad that is incapable of multiple cycles of transcription and translation of the inserted genes in human cells. The replication-defective Ad vectors can have deletions in one or more genes (or regions) involved in replication, including one or more of an E1 region, an E3 region, an E2 region, and/or an E4 region. For example, a replication-defective Ad vector can have a deletion in an E1 region, an E3 region, an E2 region, an E4 region, or a combination thereof.

The Ad can have a mutation (e.g., a deletion, insertion, inversion, or substitution) in an E1 region, an E2 region, an E3 region, and/or an E4 region. In certain embodiments, at least the E1 region and the E3 region of the Ad are deleted. In certain embodiments, at least the E1 and E3 regions are deleted, and the polynucleotide sequence of the NP is inserted in the deleted E1 region. In certain embodiments, the polynucleotide sequence encoding at least AIP-C5 is inserted in the deleted E1 region of the Ad. The polynucleotide sequence encoding at least both the NP and AIP-C5 (e.g., at least 22 amino acid residues from AIP-C5) from the CFP10 protein of Mycobacterium tuberculosis can be inserted in the deleted E1 region of the Ad.

In certain embodiments, the Ad can comprise a polynucleotide sequence that comprises a nucleic acid fragment comprising SEQ ID NO: 7 that encodes a full-length H5N1 HA comprising SEQ ID NO: 6. In certain embodiments, the Ad can comprise a polynucleotide sequence that comprises a nucleic acid fragment comprising SEQ ID NO: 9 that encodes a H5N1 HA2 with IgE comprising SEQ ID NO: 8. In certain embodiments, the Ad can comprise a polynucleotide sequence that comprises a nucleic acid fragment comprising SEQ ID NO: 11 that encodes a H5N1 M2e with IgE comprising SEQ ID NO: 10. In certain embodiments, the Ad can comprise a polynucleotide sequence that comprises a nucleic acid fragment comprising SEQ ID NO: 13 that encodes a H5N1 HA2 comprising SEQ ID NO: 12. In certain embodiments, the Ad can comprise a polynucleotide sequence that comprises a nucleic acid fragment comprising SEQ ID NO: 15 that encodes a full-length H5N1 M2e comprising SEQ ID NO: 14.

The HAd or BAd, for example, can comprise a polynucleotide sequence that comprises a nucleic acid fragment that encodes the full-length NP from H7N9 influenza virus (e.g., inserted in a deleted E1 region), or a functional fragment thereof (e.g., an immunogenic epitope). The NP can comprise SEQ ID NO: 1 and SEQ ID NO: 3. The polynucleotide sequence can comprise SEQ ID NO: 2. The NP can comprise SEQ ID NO: 1.

The polynucleotide sequence can further encode AIP-C5 from the CFP10 protein of Mycobacterium tuberculosis (e.g., at least 22 amino acid residues from AIP-C5) (e.g., inserted in a deleted E1 region). The AIP-C5 can comprise SEQ ID NO: 3. The polynucleotide sequence can comprise SEQ ID NO: 4.

In certain embodiments, the HAd or BAd expresses (e.g., includes) at least a full-length NP of an H7N9 influenza virus with or without AIP-C5 (e.g., at least 22 amino acid residues from AIP-C5) from the CFP10 protein of Mycobacterium tuberculosis, or a functional fragment thereof (e.g., an immunogenic epitope). In certain embodiments, the HAd or BAd expresses a full-length NP of an H7N9 influenza virus without the AIP-C5 from the CFP10 protein of Mycobacterium tuberculosis (e.g., HAd-NP(H7N9))

Such Ad vectors are useful for a variety of purposes. For example, such Ad vectors are useful for producing influenza antigens in vitro and in vivo (including in ovo). Accordingly, methods for generating a general immunogenicity against a heterosubtypic influenza virus in a subject are provided. In certain embodiments, a method of generating a general immunogenicity against a heterosubtypic influenza virus in a subject comprises administering to said subject an effective amount of any of the immunogenic compositions or Ads hereof. The method can provide a general immunogenicity protection (e.g., cross-protection) to the subject against various subtypes of influenza viruses (e.g., viruses selected from the group consisting of H1, H3, H5, H7, H9, and influenza B viruses).

The term “effective amount” as used herein refers to that amount of active antigen (or fragments or epitopes thereof), compound and/or pharmaceutical agent that elicits an immune response (e.g., secretory, humoral, and/or cellular protective immunity) in a subject (e.g., a mammalian subject) that is reactive with one or more targeted disease-producing viral strains. The term “protective immunity” means that a vaccine or immunization schedule that is administered to a subject induces an immune response that prevents, retards the development of, or reduces the severity of a disease that is caused by a viral strain (e.g., an influenza virus), or diminishes or altogether eliminates the symptoms of the disease. In one aspect, the effective amount is an amount of an antigen (or epitopes thereof), compound or pharmaceutical agent where there is a detectable difference between an immune response indicator measured in the subject before and after administration of a particular preparation to the subject. Immune response indicators include, without limitation, antibody titer or specificity (as detected by an assay such as enzyme-linked immunoassay (ELISA), virus-neutralization assay, hemagglutination inhibition assay, ELIspot assay, flow cytometry, immunoprecipitation, Ouchter-Lowny immunodiffusion, binding detection assays of, for example, Western blot or antigen arrays, cytotoxicity assays, and the like. However, it is to be understood that the total daily usage of the antigens and compositions described herein may be decided by the attending physician within the scope of sound medical judgment. The specific effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill. Additionally, the inclusion of AIP-C5 in the immunogenic composition can also affect the dose amount as AIP-C5 T cell response can be highly effective, thus allowing for a reduced dosage of the immunogenic composition in certain circumstances.

In certain embodiments, administration of the effective amount of the immunogenic composition or Ad can induce a dose-dependent increase in the humoral and cell-mediated immunity in the subject.

Further methods for producing influenza antigens are also provided. For example, influenza antigens can be produced by replicating an Ad that comprises at least one polynucleotide sequence that encodes SEQ ID NO: 1. For example, the influenza antigen an NP antigen selected from an H1, H3, H5, H7, H9, or influenza B strain, or a functional fragment thereof (e.g., one or more immunogenic epitopes). In some embodiments, the Ad includes sequences that encode at least SEQ ID NO: 1 and SEQ ID NO: 3. In certain embodiments, the Ad contains polynucleotide sequences that encode a plurality of influenza antigens, including without limitation SEQ ID NO: 1 or SEQ ID NOS: 1 and 3. In some embodiments, the Ad contains polynucleotide sequences that encode a plurality of influenza antigens, including without limitation SEQ ID NO: 7, SEQ ID NO: 9, SEQ ID NO: 11, SEQ ID NO: 13, or SEQ ID NO: 15.

In certain embodiments, Ad expressing influenza virus antigens are produced by introducing a HAd or BAd into a cell that can support replication of the vector. Such cells typically include at least one heterologous nucleic acid that provides a complementary replication function, such as a heterologous nucleic acid that encodes one or more E proteins that are deleted from the vector. In certain embodiments, cells that can support growth of the vector can support growth of different strains of Ad with different species tropism. Optionally, the influenza virus antigen or Ad vector is isolated, and for example, used to produce immunogenic compositions, such as vaccines.

Immunogenic compositions (e.g., vaccines) are also provided that are cross-protective against two or more subtypes of influenza viruses when administered to a subject. As used herein, the term “composition” generally refers to any product comprising more than one ingredient, including one or more influenza virus antigens produced using the Ads hereof (e.g., HAd-C5-NP(H7N9), HAd-NP(H7N9), or BAd-C5-NP(H7N9)). In certain embodiments, the immunogenic composition comprises a NP of a H7N9 influenza virus with or without expressing at least 22 amino acid residues of AIP-C5 from a CFP10 protein of Mycobacterium tuberculosis and a pharmaceutically acceptable carrier.

The NP (or functional fragment thereof) can be expressed, for example, using the HAd or BAd described herein. The NP (or functional fragment thereof) can comprise SEQ ID NO: 1. The AIP-C5 from a CFP10 protein can comprise SEQ ID NO: 3.

In certain embodiments, the immunogenic composition is cross-protective against at least five subtypes of influenza viruses when administered to a subject. For example, and without limitation, when administered to a subject, the composition can confer general immunogenicity protection against subtypes of viruses selected from the group consisting of H1, H3, H5, H7, and H9 influenza viruses. In certain embodiments, the composition is cross-protective against two or more subtypes of influenza A viruses when administered to a subject. In certain embodiments, the influenza virus(es) is/are avian influenza virus(es).

The immunogenic compositions can be administered in unit dosage forms and/or compositions containing one or more pharmaceutically acceptable carriers, adjuvants, diluents, excipients, and/or vehicles, and combinations thereof. As used herein, the term “administering” and its variants include all means of introducing the antigens and compositions described herein to the patient, including, but are not limited to, oral (p.o.), intravenous (i.v.), intramuscular (i.m.), subcutaneous (s.c.), transdermal, via inhalation (e.g., intranasal (i.n.)), buccally, intraocularly, sublingually, vaginally, rectally, and the like. The antigens and compositions described herein can be administered in unit dosage forms and/or formulations containing conventional nontoxic pharmaceutically acceptable carriers, adjuvants, and vehicles.

The term “adjuvant” refers to a substance that enhances, specifically or non-specifically, an immune response to an antigen. Non-limiting examples of adjuvants for use with the present antigens, compositions and methods include cholera toxin B subunit, flagellin, human papillomavirus L1 or L2 protein, herpes simplex glycoprotein D (gD), complement C4 binding protein, TL4 ligand, and interleukin-1 beta (IL-1β), lysolecithin, pluronic polyols, polyanions, an oil-water emulsion, dinitrophenol, iscomatrix, and liposome polycation DNA particles. The antigens and compositions hereof need not necessarily comprise an adjuvant as the HAd and BAd vectors can provide an adjuvant effect.

The immunogenic compositions can further comprise salts, for example, where the composition comprises a live vaccine (e.g., such live vaccines prepared using the Ad vectors provided herein pursuant to methodologies well-known in the art). As used herein, the term “salts” refers to buffered salts and the like as is generally known in the art. Such salts can include, for example, salts based on sodium and potassium, aluminum salts such as aluminum hydroxide, aluminum phosphate, or potassium aluminum sulphate, and/or other conventional non-toxic salts. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

The immunogenic composition can be formulated as a pharmaceutical composition and administered to a mammalian host, such as a human patient, in a variety of forms adapted to the chosen route of administration. In at least one embodiment, the immunogenic composition can be administered intranasally to a subject. In certain embodiments, the immunogenic composition is formulated to be administered subcutaneously. In certain embodiments, the immunogenic composition is formulated to be administered orally. In certain embodiments, the immunogenic composition is formulated to be administered as an aerosol spray.

In certain embodiments, the immunogenic composition is systemically administered in combination with a pharmaceutically acceptable vehicle. The percentages of the components of the compositions and preparations can vary and can be between about 1 to about 99% weight of the active ingredient(s) and a binder, excipients, a disintegrating agent, a lubricant, and/or a sweetening agent (as are known in the art). The amount of active compound (e.g., antigens) in such therapeutically useful compositions is such that an effective dosage level can be obtained.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions, which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well-known to those skilled in the art. Parenteral administration of an antigen is illustratively performed in the form of saline solutions or with the antigen incorporated into liposomes. In cases where the antigen itself is not sufficiently soluble to be dissolved, a solubilizer such as ethanol can be applied.

The pharmaceutical dosage forms suitable for injection, intranasal administration, or infusion can include sterile aqueous solutions or dispersions or sterile powders comprising the active ingredients that are adapted for the extemporaneous preparation of sterile injectable or infusible solutions or dispersions, optionally encapsulated in liposomes, nanocrystals, or polymeric nanoparticles. In all cases, the ultimate dosage form should be sterile, fluid, and stable under the conditions of manufacture and storage. The liquid carrier or vehicle can be a solvent or liquid dispersion medium comprising, for example and without limitation, water, electrolytes, sugars, ethanol, a polyol (e.g., glycerol, propylene glycol, liquid polyethylene glycols, and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants.

Sterile injectable solutions can be prepared by incorporating the immunogenic compositions in the required amount of the appropriate solvent with one or more of the other ingredients set forth above, as required, followed by filter sterilization. In the case of sterile powders for the preparation of sterile injectable solutions, the preferred methods of preparations are vacuum drying and the freeze-drying techniques, which yield a powder of the active ingredient plus any additional desired ingredient present in the previously sterile-filtered solutions.

The dosage depends on several factors, including: the administration method, the targeted disease-producing viral strain, the severity of the subject's present condition where an active infection exists, whether an active infection exists to be treated, or the vaccination is prophylactic, and the age, weight, and health of the subject. Additionally, pharmacogenomic (the effect of genotype on the pharmacokinetic, pharmacodynamic or efficacy profile of the antigen or composition) information about a particular patient may affect the dosage used.

For methods described herein, the antigens and compositions can be administered in a single dose, or via a combination of multiple dosages, which can be administered by any suitable means, contemporaneously, simultaneously, sequentially, or separately. Where the dosages are administered in separate dosage forms, the number of dosages administered per day for each antigen or composition can be the same or different. The antigen and/or composition dosages can be administered via the same or different routes of administration. The antigens or compositions can be administered according to simultaneous or alternating regimens, at the same or different times during the course of the therapy, concurrently in divided or single forms.

Depending upon the route of administration, a wide range of permissible dosages are contemplated herein, including doses falling in the range from about 10⁶ to 1011 virus particles (VP)/kg. The dosages may be single or divided and may be administered according to a wide variety of protocols, including q.d. (once a day), b.i.d. (twice a day), t.i.d. (three times a day), or even every other day, once a week, once a month, once a quarter, and the like. In each of these cases it is understood that the effective amounts described herein correspond to the instance of administration, or alternatively to the total daily, weekly, month, or quarterly dose, as determined by the dosing protocol.

In addition to the illustrative dosages and dosing protocols described herein, an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### Certain Definitions

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range. The term “substantially” can allow for a degree of variability in a value or range, for example, within 90%, within 95%, 99%, 99.5%, 99.9%, 99.99%, or at least about 99.999% or more of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be“acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “patient” and “subject” are used interchangeably and include a human patient, a laboratory animal, such as a rodent (e.g., mouse, rat, or hamster), a rabbit, a monkey, a chimpanzee, a domestic animal, such as a dog, a cat, or a rabbit, an agricultural animal, such as a cow, a horse, a pig, a sheep, or a goat, or a wild animal in captivity, such as a bear, a panda, a lion, a tiger, a leopard, an elephant, a zebra, a giraffe, a gorilla, a dolphin, or a whale. The patient to be treated is preferably a mammal, in particular a human being.

The terms “cell” and “cell culture” are used interchangeably and all such designations include progeny. It is understood that all progeny may not be precisely identical in DNA content, due to deliberate or inadvertent mutations. Mutant progeny that have the same function or biological activity as screened for in the originally transformed cell are included.

The term “gene” refers to a functional protein, polypeptide, or peptide-encoding nucleic acid unit (e.g., the ectodomains of influenza A Matrix Protein 2 (M2e) and a stem region of an influenza A hemagglutinin 2 (HA2) encoding nucleic acids. As will be understood by those in the art, this functional term includes genomic sequences, cDNA sequences, probes, oligonucleotides or fragments thereof (and combinations thereof), as well as gene products including those that have been designed and/or altered by a user. Purified genes, nucleic acids, protein and the like are used to refer to these entities when identified and separated from at least one contaminating nucleic acid or protein with which it is ordinarily associated.

The term “immunization” refers to the process of inducing a continuing protective level of antibody and/or cellular immune response that is directed against an influenza antigen (or fragment thereof), either before or after exposure of the host to the influenza strain.

The term “immunogen” or “immunogenic” refers to an antigen that is capable of initiating lymphocyte activation resulting in an antigen-specific immune response. An immunogen therefore includes any molecule that contains one or more epitopes that will stimulate a host's immune system to initiate a secretory, humoral, and/or cellular antigen-specific response.

The terms “protein,” “polypeptide” and “peptide” refer to compounds comprising amino acids joined via peptide bonds and are used interchangeably.

The term “vector” is used in reference to nucleic acid molecules that transfer DNA segment(s) from one cell to another. The term “vehicle” is sometimes used interchangeably with “vector.” The term “vector” as used herein also includes expression vectors in reference to a recombinant DNA molecule containing a desired coding sequence and appropriate nucleic acid sequences necessary for the expression of the operably linked coding sequence in a particular host organism. Nucleic acid sequences necessary for expression in prokaryotes or eukaryotes usually include a promoter, an operator (optional), and a ribosome binding site, often along with other sequences. Eukaryotic cells are known to use promoters, enhancers, and termination and polyadenylation signals. The term “vector” can be used to described the use of a carrier or other delivery system or organism to deliver the antigen(s) hereof to a host to trigger an immune response as part of a vaccine. Non-limiting examples of these vaccine vectors include viruses, bacteria, protozoans, cells (e.g., homologous or heterologous), and the like which can be live, live-attenuated, heat-killed, mechanically-killed, chemically-killed, or recombinant (e.g., peptides, proteins and the like) as is known to those skilled in the art of vaccine preparation. The skilled artesian will readily recognize the type of “vector” to which this specifications and claims refer based on the description of the materials and methods used and described herein.

### EXAMPLES

The following examples serve to illustrate the present disclosure. The examples are not intended to limit the scope of the claimed invention.

### Materials

HEK293 (human embryonic kidney cells expressing HAdV-C5 E1 proteins), 293Cre (293 cells expressing Cre recombinase), BHH2C (bovine-human hybrid clone 2C), and MDCK (Madin-Darby canine kidney) cell lines were grown as monolayer cultures in Corning™ Dulbecco's Modification of Eagle's Medium (DMEM) (Fisher Scientific, Hampton, NH) containing either 10% reconstituted fetal bovine serum (Hyclone, Logan, UT) and gentamycin (50 μg/ml). See Graham et al., Characteristics of a human cell line transformed by DNA from human adenovirus type 5, J Gen Virol 36: 59-74, doi:10.1099/0022-1317-36-1-59 (1977); Chen et al., Production and characterization of human 293 cell lines expressing the site-specific recombinase Cre, Somatic Cell & Mol Gen 22: 477-488 (1996); van Olphen and Mittal, Development and characterization of bovine x human hybrid cell lines that efficiently support the replication of both wild-type bovine and human adenoviruses and those with E1 deleted, J Virol 76: 5882-5892 (2002).

The nucleoprotein (NP) gene of the A/Anhui/l/2013(H7N9) influenza virus without [NP(H7N9)] or with AIP-C5 [C5-NP(H7N9)] was synthesized commercially (GenScript Biotech Corporation, Piscataway, NJ). The NP(H7N9) or C5-NP(H7N9) under the control of the cytomegalovirus (CMV) promoter and bovine growth hormone (BGH) polyadenylation signal were inserted into the HAd E1 shuttle plasmid. The vectors [HAd-NP(H7N9) and HAd-C5-NP(H7N9)] were generated following a Cre-recombinase-mediated site-specific recombination technique. See Sayedahmed et al., Current use of adenovirus vectors and their production methods, In Viral Vectors for Gene Therapy: Methods and Protocols, Manfredsson, F. P, Benskey, M. J., Eds., Springer New York: New York, NY: 155-175 (2019).

HAd-ΔE1E3 (HAd-5 E1 and E3 deleted empty vector) was prepared as described in Noblitt et al., Decreased tumorigenic potential of EphA2-overexpressing breast cancer cells following treatment with adenoviral vectors that express EphrinAl, Cancer Gene Ther 11: 757-766, doi:10.1038/sj.cgt.7700761 (2004). HAd-NP(H7N9) and HAd-C5-NP(H7N9), and HAd-ΔE1E3 were grown in 293 cells and titrated in BBH2C cells as described in Vemula (2013), supra.

For immunization studies, the vectors were purified by cesium chloride density gradient ultracentrifugation following a published protocol. (see Pandey et al., Impact of preexisting adenovirus vector immunity on immunogenicity and protection conferred with an adenovirus-based H5N1 influenza vaccine, PLoS One 7: e33428, doi:10.1371/journal.pone.0033428 (2012)).

A/Puerto Rico/8/1934(H1N1), A/Hong Kong/1/68(H3N2), A/chukkar/MN/14951-7/1998(H5N2), A/goose/Nebraska/17097/2011(H7N9), or A/Hong Kong/1073/1999(H9N2) were grown in embryonated hen eggs and titrated in the eggs and/or MDCK.

The statistical significance was set at p<0.05 where applicable. Two-way ANOVA with Bonferroni post-test was used to ascertain statistical significance where appropriate.

### Example 1

### Generation of HAd-NP(H7N9) and HAd-C5-NP(H7N9) Vectors

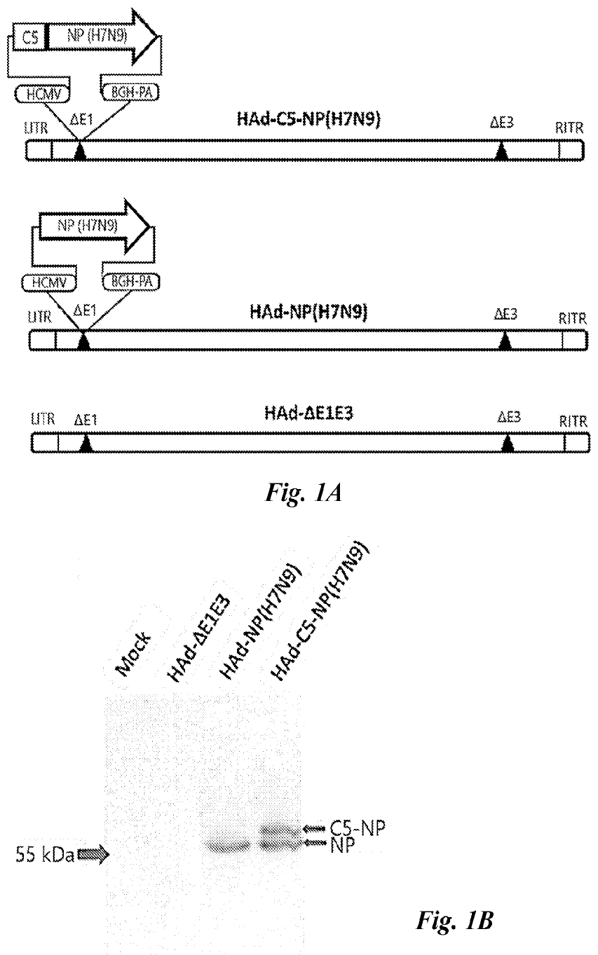

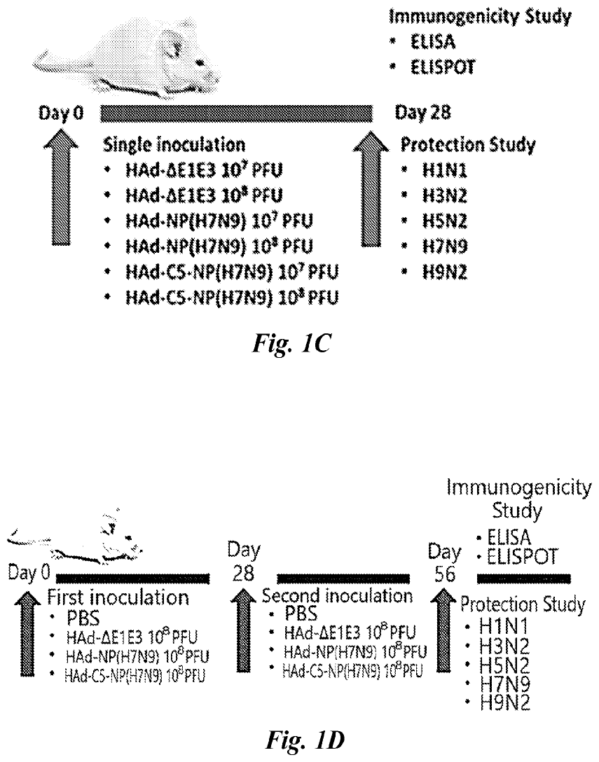

The HAd vectors [HAd-NP(H7N9) and HAd-C5-NP(H7N9)] containing the H7N9 NP gene of the A/Anhui/1/2013(H7N9) influenza A virus with or without AIP-C5 were generated (FIG. 1A) by the Cre recombinase-mediated homologous recombination. (see Anton and Graham, Site-specific recombination mediated by an adenovirus vector expressing the Cre recombinase protein: a molecular switch for control of gene expression. J of Virology 69: 4600-4606, doi:10.1128/JVI.69.8.4600-4606.1995 (1995)). The presence of the foreign gene cassette in the vector was identified initially by restriction analysis followed by sequencing the region containing the gene cassette. To confirm the expression of NP or C5-NP in 293 cells infected with HAd-NP(H7N9) or HAd-C5-NP(H7N9)], vector-infected cell extracts were processed for immunoblot assay using an NP-specific mouse monoclonal antibody. Mock-infected or HAd-ΔE1E3 (empty vector)-infected cell extracts were used as negative controls.

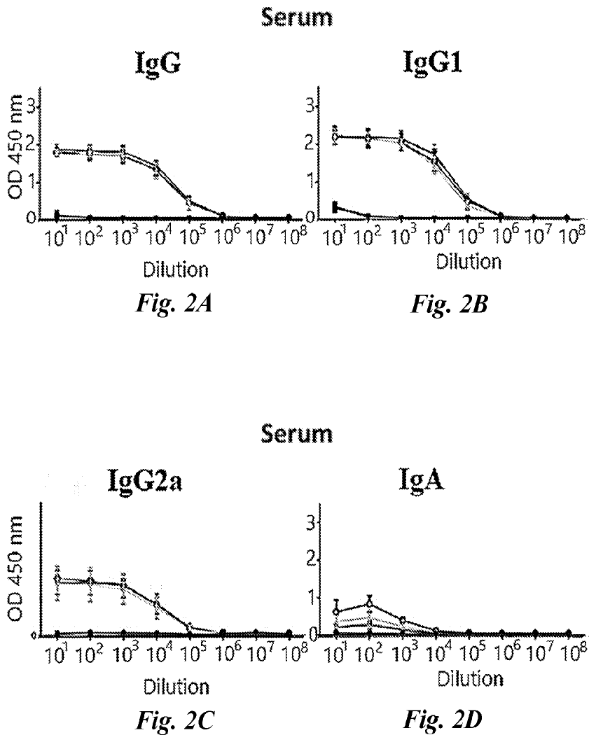

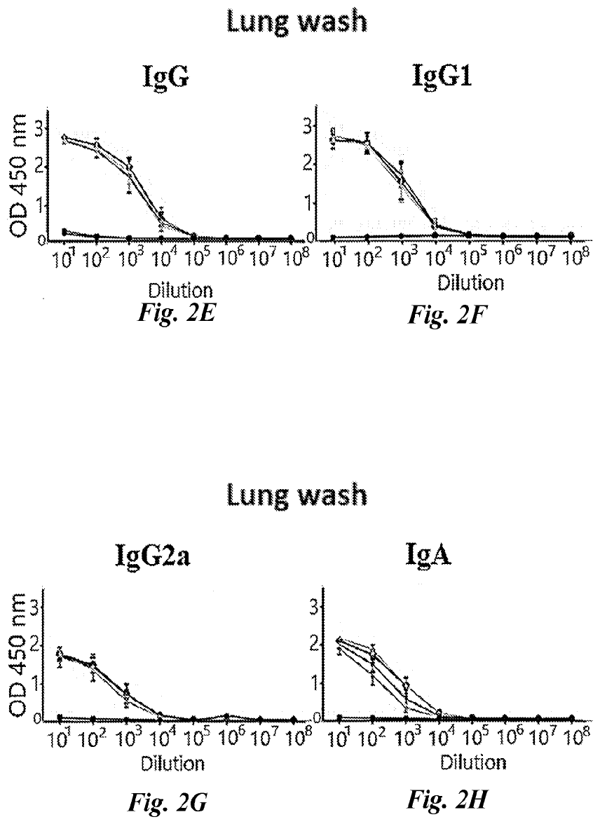

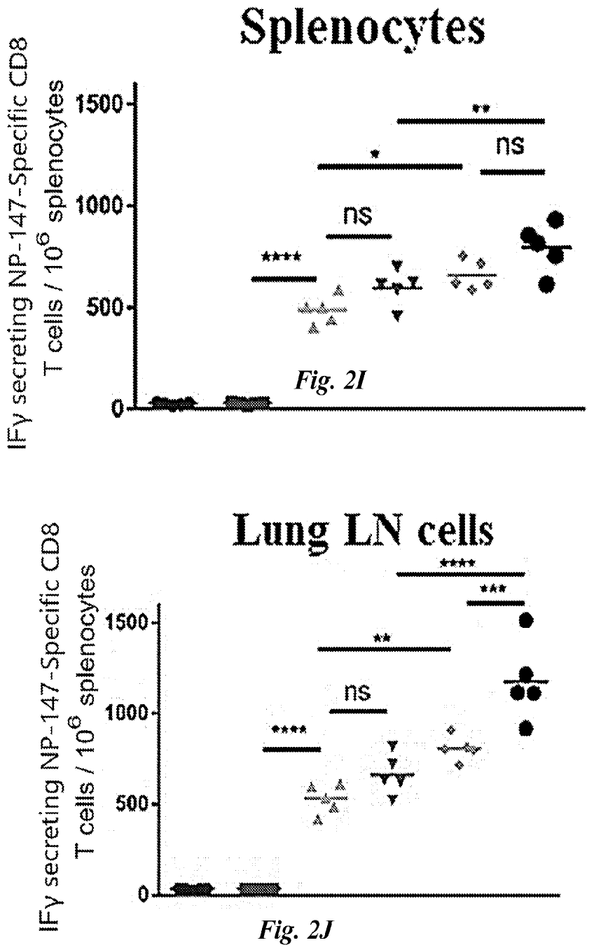

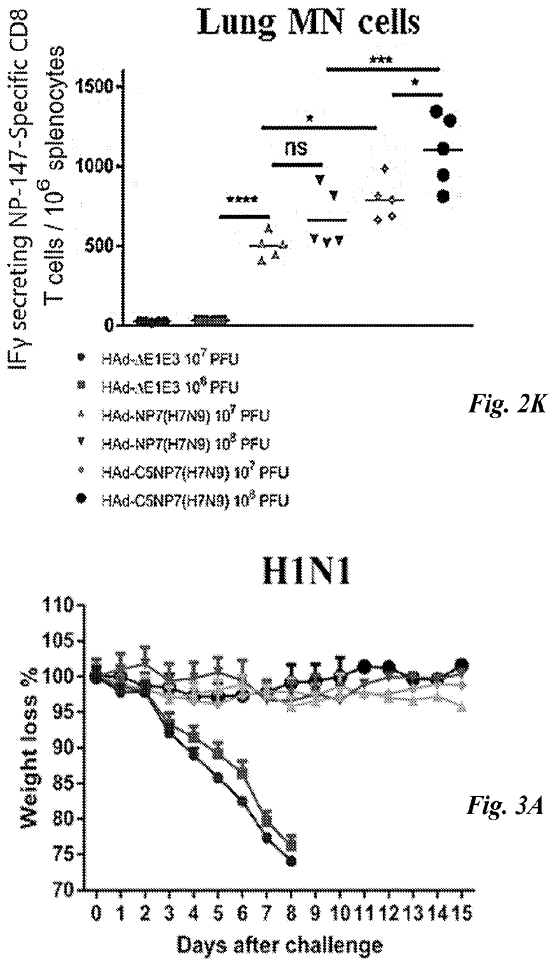

The presence of an approximately 56 kDa band with HAd-NP(H7N9)-infected cell extract or two bands of about 56 and 61 kDa with HAd-C5-NP(H7N9)]-infect cell extract supports the expression of NP or C5-NP, respectively (FIG. 2B).

### Example 2

Development of Similar Levels of Humoral Immune Responses in Mice Immunized i.n. With HAd-NP(H7N9) or HAd-C5-NP(H7N9)

All studies were performed in a BSL-2+ facility with the approvals of the Institutional Biosafety Committee (IBC) and the Institutional Animal Care and Use Committee (IACUC) using six-to-eight-week-old BALB/c mice (Jackson Laboratory). The overall experimental design for the one-dose regimen is outlined in FIG. 1C.

NP-specific antibodies are not considered as virus-neutralizing; however, NP-specific antibody-dependent cell-mediated cytotoxicity (ADCC) and complement-dependent lysis (CDL) have been observed. (see Jegaskanda et al., Induction of H7N9-Cross-Reactive ADCC antibodies by human seasonal Influenza A viruses that are directed toward the nucleoprotein. J Infect Dis 215: 818-823, doi:10.1093/infdis/jiw629 (2017)).

The BALB/c mouse groups were vaccinated intranasally (i.n.) once with 1×10⁷ or 1×10⁸ plaque-forming units (PFU) of HAd-NP(H7N9), HAd-C5-NP(H7N9), or HAd-ΔE1E3. Specifically, the animals (10 animal/group) were mock-inoculated (PBS) or inoculated i.n. once or twice (at a 3-week interval) with 1×10⁸ PFU of HAd-NP(H7N9), HAd-C5-NP(H7N9), or HAd-ΔE1E3. For the single-dose regimen, animal groups were also vaccinated i.n. with 1×10⁷ PFU of HAd-NP(H7N9), HAd-C5-NP(H7N9), or HAd-ΔE1E3.

Four-week post-inoculation (single-dose regimen) or three weeks post-booster inoculation (two-dose regimen), 5 animals/group were anesthetized, the blood samples were obtained via retro-orbital puncture, and the lung washes were attained by homogenizing one lung from each animal in 1 ml of PBS as described in Papp et al., Mucosal immunization with recombinant adenoviruses: induction of immunity and protection of cotton rats against respiratory bovine herpesvirus type 1 infection, J Gen Virol 78(11): 2933-2943, doi:10.1099/0022-1317-78-11-2933 (1997). The serum samples and lung washes were utilized to assess the development of humoral immune responses. The second lung was processed to collect the lung MN cells using MagniSort® Mouse CD3 Positive Selection Kit (Affymetrix eBioscience San Diego, CA) and used to evaluate cell-mediated immunity (CMI) responses. The spleens and mediastinal lymph nodes (LNs) were also collected to determine CMI responses.

The remaining five animals per group were challenged i.n. with 2 lethal dose 50 (LD₅₀) of A/Puerto Rico/8/1934(H1N1), 5 LD₅₀ of A/Hong Kong/1/68(H3N2), or 100 mouse infectious dose 50 (MID₅₀) of A/chukkar/MN/14951-7/1998(H5N2), A/goose/Nebraska/17097/2011(H7N9), or A/Hong Kong/1073/1999(H9N2). For the lethal challenge, animals were monitored daily for morbidity and mortality for two weeks post-challenge. Whereas for the nonlethal challenge, the lungs were collected on Day 3 post-challenge, and viral titers were determined in MDCK or embryonated chicken eggs. (see Vemula (2013), supra).

None of the serum samples had any detectable hemagglutination inhibition (HI) or virus-neutralizing (VN) antibody titers against an H7N9 influenza virus (data not shown). Low levels of NP-specific IgA as well as very high levels of NP-specific IgG, IgG1, and IgG2a were detected in sera of mouse groups immunized either with HAd-NP(H7N9) or HAd-C5-NP(H7N9) (FIGS. 2A-2D). Both the HAd-NP(H7N9) and HAd-C5-NP(H7N9) groups showed similar levels of humoral immune responses in the serum samples, indicating that the inclusion of AIP-C5 did not have significant impact on the levels of systemic humoral immune responses. The control groups inoculated i.n. with HAd-ΔE1E3 did not induce anti-NP humoral immune response levels above background (FIGS. 2A-2D). No significant dose-dependent differences in humoral immune responses were observed in vaccinated animals.

The development of NP-specific humoral immune responses at the mucosal level was also determined. An enzyme-linked immunosorbent assay (ELISA) was performed as described in Mittal et al., Pathogenesis and immunogenicity of bovine adenovirus type 3 in cotton rats (Sigmodon hispidus), Virology 213: 131-139, doi:10.1006/viro.1995.1553 (1995); and Mittal et al., Immunization with DNA, adenovirus or both in biodegradable alginate microspheres: effect of route of inoculation on immune response, Vaccine 19: 253-263 (2000). Briefly, 96-well ELISA plates (eBioscience, San Diego, CA) were coated with purified NP protein (0.5 μg/ml) of H7N9 (MyBioSource, Inc., San Diego, CA, USA) and incubated overnight at 4° C. After blocking with 1% bovine serum albumin (BSA) in PBS, diluted serum samples (1:500 for IgG & IgG1 and 1:50 for IgG2a) or lung washes (1:10) were added and incubated at room temperature for 2 hours. The horseradish peroxidase-conjugated goat anti-mouse IgG, IgG1, IgG2a, IgG2b, or IgA antibodies (Invitrogen, Waltham, MA and Thermo Fisher Scientific Corporation, Waltham, MA) at a suggested dilution for each antibody was added and incubated at room temperature for 2 hours. ABD OptEIA™ ELISA sets TMB substrate (Thermo Fisher Scientific Corporation, Waltham, MA) was used for color development. The reaction was stopped with 2N sulfuric acid solution, and the optical density readings were obtained at 450 nm using a SpectraMax® i3x microplate reader (Molecular Devices, LLC, Sunnyvale, CA).

High levels of NP-specific IgA, IgG, IgG1, and IgG2a were observed in the lung washes of mouse groups immunized either with HAd-NP(H7N9) or HAd-C5-NP(H7N9) (FIGS. 2E-2H). Both the HAd-NP(H7N9) and HAd-C5-NP(H7N9) groups showed similar levels of humoral immune responses in the lung washes. The lung washes collected from the control groups inoculated i.n. with HAd-ΔE1E3 did not elicit anti-NP humoral immune responses above background (FIGS. 2E-2H). Again, there were no obvious dose-dependent differences in NP-specific mucosal immune responses in the vaccinated groups.

### Example 3

### Enhancement of NP-Specific CD8 T Cell Responses by HAd-C5-NP(H7N9) Compared to HAd-NP(H7N9)

The influenza virus internal protein NP is conserved across multiple subtypes and serves as a robust inducer of CTLs and non-neutralizing antibody responses. (see Laidlaw et al., Cooperativity between CD8+ T cells, non-neutralizing antibodies, and alveolar macrophages is important for heterosubtypic influenza virus immunity, PLoS Pathog 9: e1003207, doi:10.1371/journal.ppat.1003207 (2013)). NP-specific CD8 T cell responses are vital for the influenza virus clearance following infection and perform a critical role in homologous and heterosubtypic protection against influenza viruses. (see Yewdell et al., Influenza A virus nucleoprotein is a major target antigen for cross-reactive anti-influenza A virus cytotoxic T lymphocytes, Proc Nat Acad Sci USA 82: 1785-1789 (1985); and Taylor and Askonas, Influenza nucleoprotein-specific cytotoxic T-cell clones are protective in vivo. Immunology, 58: 417-420 (1986)). In addition, AIP-C5 has been shown to enhance CMI responses due to antigen processing through autophagy. (see Khan et al., A novel bovine adenoviral mucosal vaccine expressing a Mycobacterium tuberculosis antigen-85B epitope and an autophagy-inducing peptide protects mice against tuberculosis through robust pulmonary and systemic immune responses, Cell Rep Med 2: 100372 (2021)). To investigate the impact of AIP-C5 on augmentation of CD8 T cell responses in the HAd-C5-NP(H7N9) group compared to the HAd-NP (H7N9 group, splenocytes, mediastinal LN cells, and lung mononuclear (MN) cells were collected to monitor the development of CD8 T cell responses using ELISpot assays.

The interferon gamma (INF-7) ELISpot assay was performed as described in Hoelscher et al., Development of adenoviral-vector-based pandemic influenza vaccine against antigenically distinct human H5N1 strains in mice, Lancet 367: 475-481, doi:10.1016/S0140-6736(06)68076-8 (2006). The splenocytes, mediastinal LN, and lung MN cells were stimulated with the NP147 [TYQRTRALV (SEQ ID NO: 5)] peptide (H-2K^{d}-restricted CTL epitope for NP), and stimulated cells were processed for INFγ ELISpot assay. (see Rotzschke et al., Isolation and analysis of naturally processed viral peptides as recognized by cytotoxic T cells, Nature 348: 252-254, doi:10.1038/348252a0 (1990)). The number of spots forming units (SFU) were enumerated using AID iSpot Advanced Imaging Device (Autoimmun Diagnostika GmbH, Strassberg, Germany).

There was a significantly higher number of NP-specific IFN-γ secreting CD8 T cells in the spleen (FIG. 2I), mediastinal LN (FIG. 2J), and lung MN cells (FIG. 2K) in the HAd-C5-NP(H7N9) group as compared to the HAd-NP(H7N9) group, supporting that the AIP-C5 led to the enhancement of CD8 T cell responses. Also, there were dose-dependent increases in the CMI responses in vaccinated groups.

### Example 4

Protection of Mouse Groups Immunized with HAd-C5-NP(H7N9) Compared to HAd-NP(H7N9) Following Challenge with H1N1, H3N2, H5N2, H7N9, and H9N2 Influenza a Viruses

To determine homo- and hetero-subtypic protection, HAd-C5-NP(H7N9) or HAd-NP(H7N9) immunized mouse groups were challenged i.n. with 2 lethal dose 50 (LD₅₀) of A/Puerto Rico/8/1934(H1N1), 5 LD₅₀ of A/Hong Kong/1/68(H3N2), 100 mouse infectious dose 50 (MID₅₀) of A/chukkar/MN/14951-7/1998(H5N2), A/goose/Nebraska/17097/2011(H7N9), or A/Hong Kong/1073/1999(H9N2). Since A/Puerto Rico/8/1934(H1N1) or A/Hong Kong/1/68(H3N2) influenza virus causes morbidity or mortality in mice, the vaccine efficacy was evaluated by monitoring morbidity or mortality in mice for two weeks following challenge. Whereas, A/chukkar/MN/14951-7/1998(H5N2), A/goose/Nebraska/17097/2011(H7N9), or A/Hong Kong/1073/1999(H9N2) do not induce morbidity or mortality in mice, significant reductions in lung viral titers in vaccinated animals following challenge were considered as a parameter of the vaccine protective efficacy.

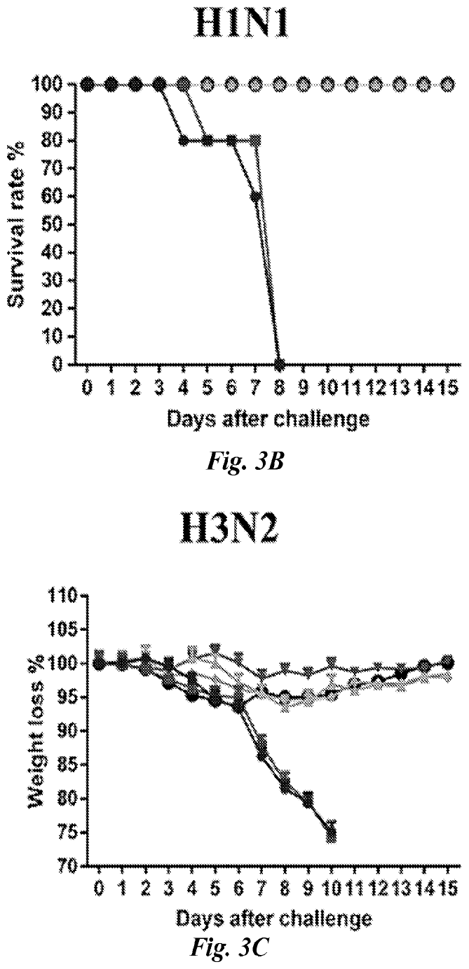

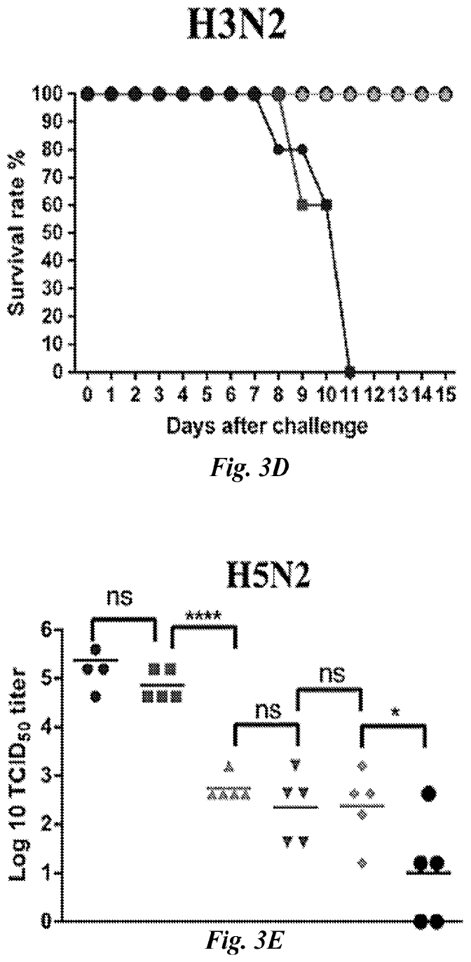

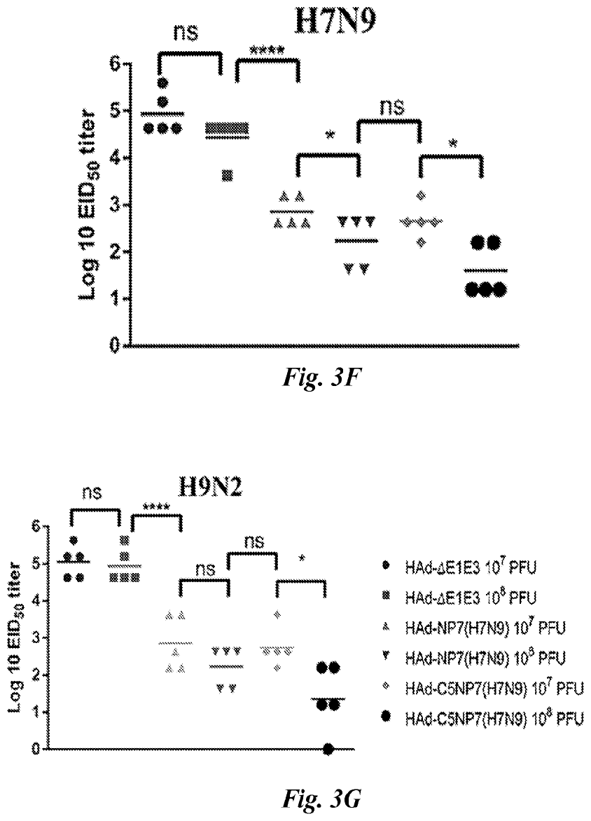

Both HAd-C5NP(H7N9) and HAd-NP(H7N9) immunized mouse groups with 10⁷ or 10⁸ PFU vaccine dose were protected from significant morbidity or mortality following challenge with A/Puerto Rico/8/1934(H1N1) (FIG. 3A and FIG. 3B) or A/Hong Kong/1/68(H3N2) (FIG. 3C and FIG. 3D). However, HAd-C5NP(H7N9) immunized groups either with 10⁷ or 10⁸ PFU provided significantly better protected following challenge with A/chukkar/MN/14951-7/1998 (H5N2) (FIG. 3E), A/goose/Nebraska/17097/2011 (H7N9) (FIG. 3F) or A/Hong Kong/1073/1999 (H9N2) (FIG. 3G) as compared to the HAd-NP(H7N9) vaccinated groups. These results support that the AIP-C5-dependent augmentation of NP-specific CMI response confers enhanced heterosubtypic protection.

### Example 5

### Efficacy of Two-Dose Regimen of HAd-C5NP(H7N9) or HAd-NP(H7N9) in Eliciting Heterosubtypic P Protection

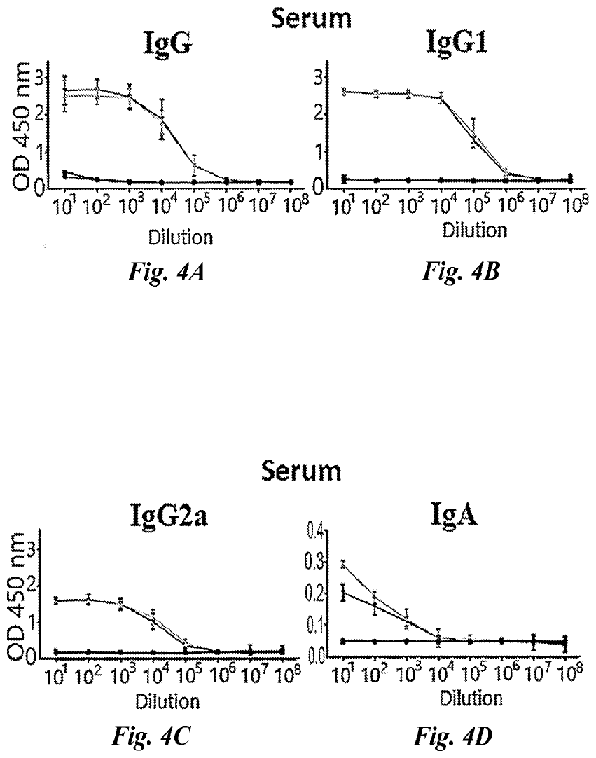

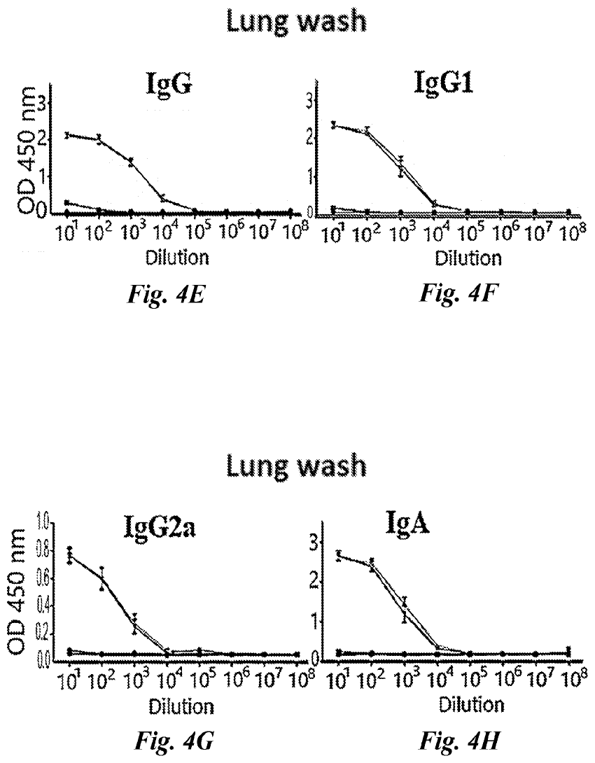

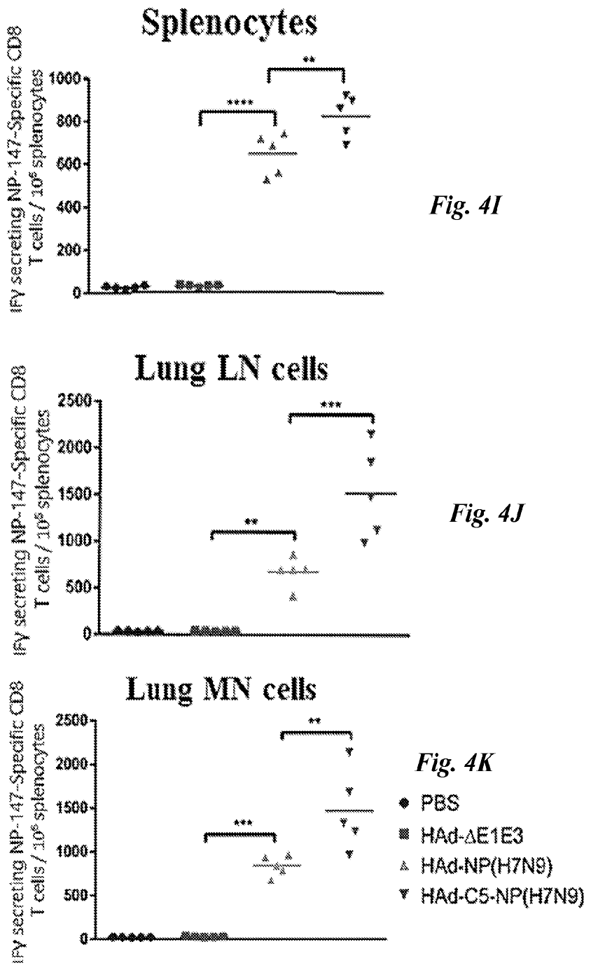

To determine whether the protection efficacy HAd-C5NP(H7N9) or HAd-NP(H7N9) could be further improved against H5N2, H7N9 and H9N2, the immunogenicity and challenge studies were repeated with a two-dose regimen of i.n. immunization with 1×10⁸ PFU of either HAd-C5NP(H7N9) or HAd-NP(H7N9) (FIG. 1D). Similar levels of NP-specific IgG, IgG1, IgG2a, and IgA antibodies in serum samples (FIGS. 4A-D) or lung wash (LW) (FIGS. 4E-H) were observed in groups immunized either with HAd-C5NP(H7N9) or HAd-NP(H7N9). As expected, there was a significantly higher number of NP-specific IFN-γ secreting CD8 T cells in the spleen (FIG. 4I), mediastinal LN (FIG. 4J), and lung MN cells (FIG. 4K) in the HAd-C5-NP(H7N9) group as compared to the HAd-NP(H7N9) group, indicating the role of AIP-C5 in eliciting enhanced CD8 T cell responses.

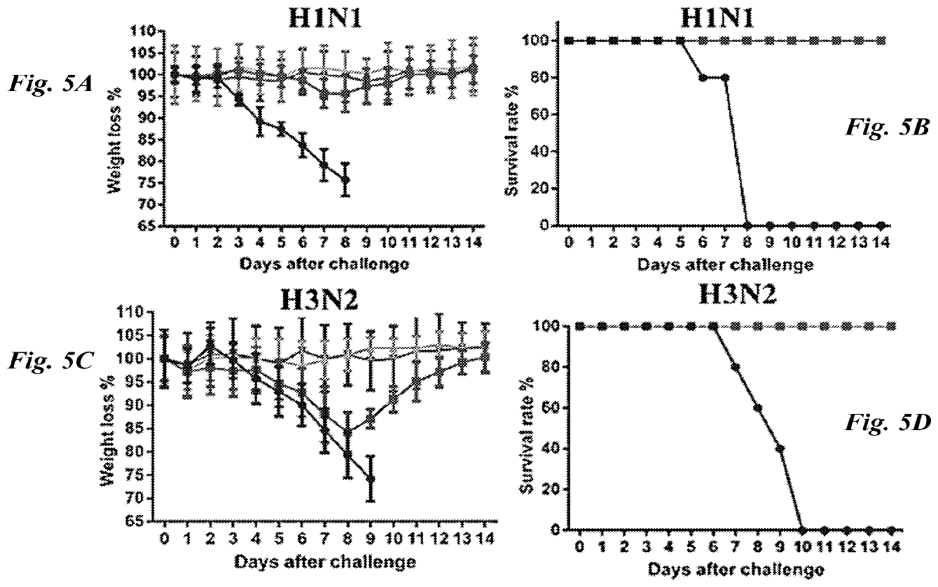

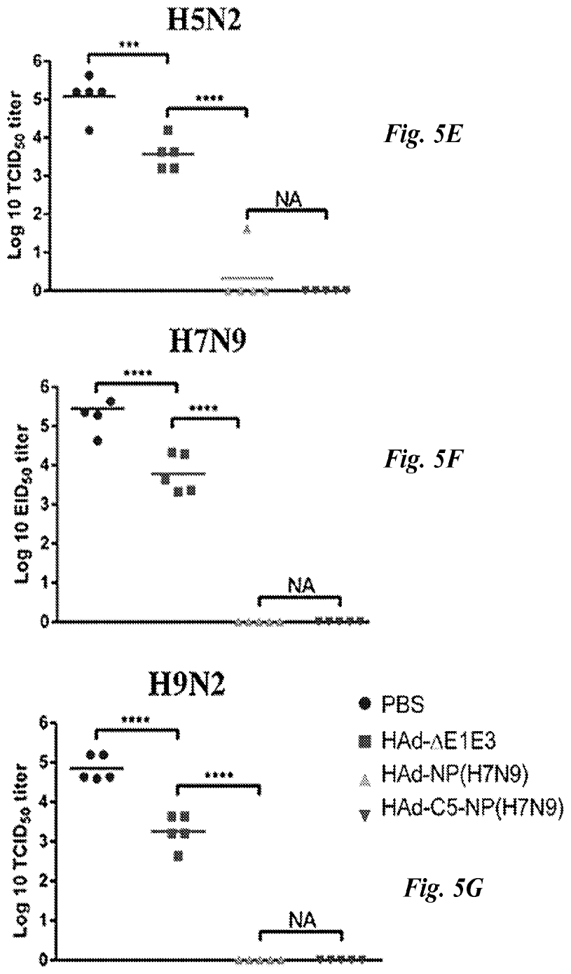

Both HAd-C5NP(H7N9) and HAd-NP(H7N9) immunized mouse groups were fully protected from morbidity or mortality following challenge with A/Puerto Rico/8/1934(H1N1) (FIGS. 5A and 5B) or A/Hong Kong/1/68(H3N2) (FIGS. 5C and 5D) influenza virus. Both HAd-C5NP(H7N9) and HAd-NP(H7N9) vaccinated mouse groups conferred complete protected following challenge with A/chukkar/MN/14951-7/1998(H5N2) (FIG. 5E), A/goose/Nebraska/17097/2011(H7N9) (FIG. 5F) or A/Hong Kong/1073/1999(H9N2) (FIG. 5G) influenza virus except one animal showed a detectable lung virus titer in the HAd-NP(H7N9)-immunized group challenged with H5N2. Animals immunized either with HAd-C5NP(H7N9) or HAd-NP(H7N9) were also challenged with a lethal A/Anhui/1/2013(H7N9) influenza virus and were fully protected from morbidity and mortality (data not shown). Overall, the two-dose regimen support that enhanced heterosubtypic protection can be achieved with as an NP-based mucosal vaccine.

Compared to phosphate buffered saline (PBS) groups, in HAd-ΔE1E3 (empty vector)-inoculated groups, there was a significant decline in morbidity with no mortality following challenge with A/Puerto Rico/8/1934(H1N1) (FIGS. 5A and 5B) or A/Hong Kong/1/68(H3N2), (FIGS. 5C and 5D) influenza virus. Similarly, in HAd-ΔE1E3-inoculated groups, there was a significant decrease in the lung virus titers following challenge with A/chukkar/MN/14951-7/1998 (H5N2) (FIG. 5E), A/goose/Nebraska/17097/2011 (H7N9) (FIG. 5F) or A/Hong Kong/1073/1999 (H9N2) (FIG. 5G) influenza virus. This phenomenon was mainly due to the development of innate lymphoid cells by mucosal immunization with HAd-ΔE1E3, as documented in detail elsewhere. (see Mittal et al., Method and composition against virus infections with activated innate lymphoid cells (ILCs), U.S. Patent Application Publication No. 2022/0062357 filed Aug. 24, 2021).

### Example 6

Lung Histopathology of Mice Immunized i.n. With HAd-NP(H7N9) or HAd-C5-NP(H7N9)

Since autophagy is a natural mechanism of removing cellular debris to improve cell functioning, the inclusion of AIP-C5 with NP should not impact the inflammatory responses. To address this, mouse groups were mock-inoculated or immunized with HAd-ΔE1E3, HAd-NP(H7N9), or HAd-C5-NP(H7N9), at various times post-inoculation, the animals were euthanized, and the lung samples were collected and processed for histopathology. Specifically, BALB/C mice (3 animal/group) were mock-immunized (PBS) or immunized i.n. with 10⁸ PFU of HAd-ΔE1E3, HAd-NP(H7N9), or HAd-C5-NP(H7N9) at 1, 2, 4, and 8 days, the animals were euthanized, and the lung were collected. The tissue samples were processed for histopathology at the Histology Research Laboratory, Center for Comparative Translational Research, Purdue College of Veterinary Medicine (West Lafayette, IN). The tissue section slides were examined and graded for histopathological lesions by a board-certified veterinary pathologist, who was not involved with the study design.

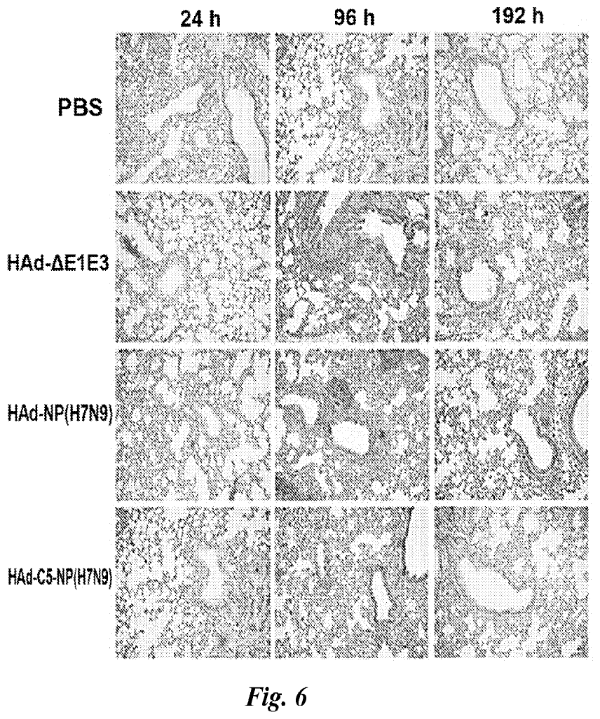

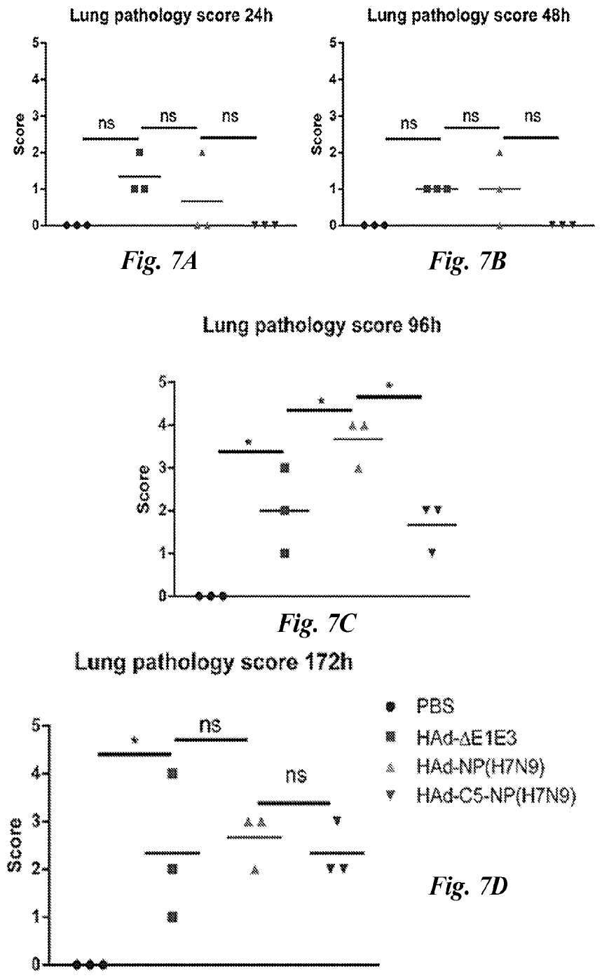

No noticeable differences in the lung histology were observed in mice immunized with HAd-C5-NP(H7N9) at any time points (FIGS. 6-7D), suggesting that the inclusion of AIP-C5 with NP did not lead to the enhancement of inflammatory responses.

### Example 7

Autophagy RT² Profiler™ PCR Array for Mice Immunized i.n. With HAd-NP(H7N9) or HAd-C5-NP(H7N9)

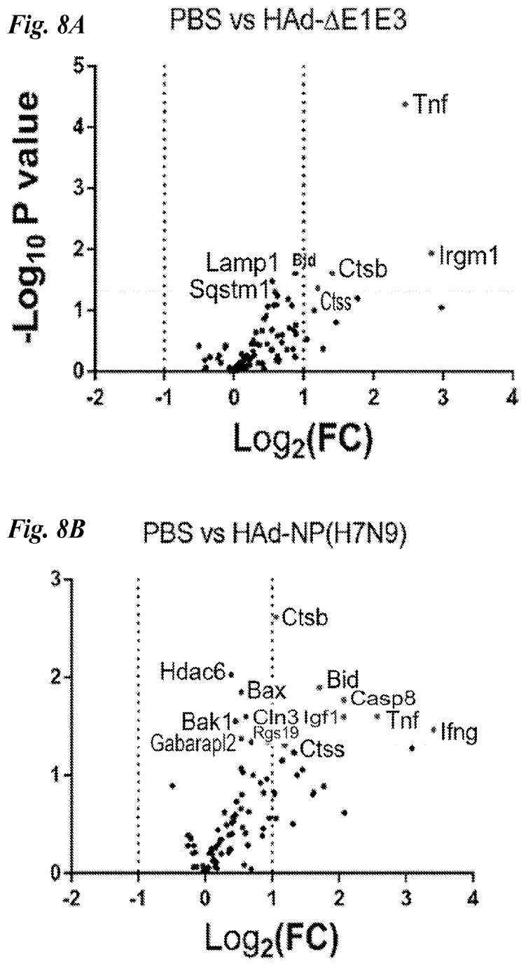

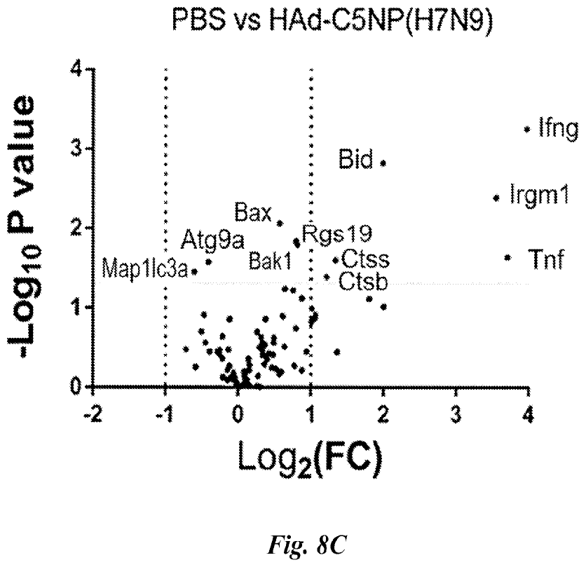

To determine the changes in autophagy-related gene expression, mouse groups were mock-inoculated or immunized with PBS, HAd-ΔE1E3, HAd-NP(H7N9), or HAd-C5-NP(H7N9), at 24 hours post-inoculation, the animals were euthanized, and the lung samples were collected and processed for ribonucleic acid (RNA) extraction. Specifically, BALB/C mice (3 animals/group) were mock-immunized (PBS) or immunized i.n. with 10⁸ PFU of HAd-ΔE1E3, HAd-NP(H7N9), or HAd-C5-NP(H7N9). At 24 hours post-inoculation, the animals were euthanized, the lungs were collected, and the lung tissue samples were processed for RNA extraction. RNA samples were used for Autophagy RT² Profiler™ PCR Array (QIAGEN Sciences Inc., Germantown, MD). The Volcano Plots identified significant gene expression changes in lung samples from HAd-NP(H7N9)- or HAd-C5-NP(H7N9)-infected animals as compared with the PBS control (FIGS. 8A-8C).

### Example 8

### Preparation of BAd3 Vectors

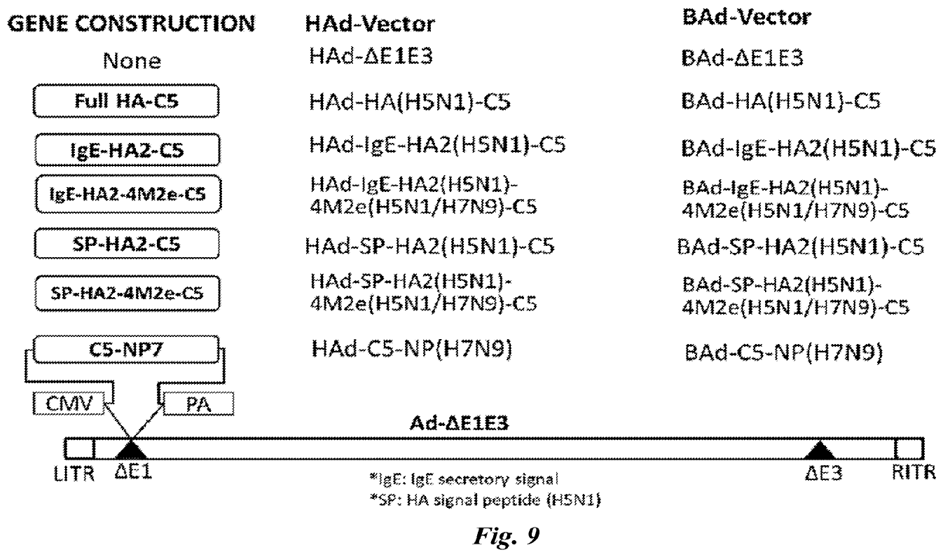

For the generation of replication-defective BAd3 vectors, a human-bovine hybrid cell line expressing Ad E1 (BHH3-BE1BF5 (BHH-F5)) was developed and adapted for use with homologous recombination in bacteria. (see van Olphen and Mittal, Generation of infectious genome of bovine adenovirus type 3 by homologous recombination in bacteria, J Virol Methods 77: 125-129 (1999), and Singh et al., Bovine adenoviral vector-based H5N1 influenza vaccine overcomes exceptionally high levels of pre-existing immunity against human adenovirus, Mol Ther 16: 965-971 (2008)). Some of the BAd vectors were generated by I-SceI recombination system using BHH-F5 expressing I-SceI (BHH3-BE1BF5/I-SceI (BHH-F5/I-SceI)). The names of BAd vectors expressing the immunogenic proteins of influenza with AIP-C5 are shown in FIG. 9.

The presence of the foreign gene cassettes in the BAd vaccine platform were initially identified by restriction analysis followed by sequencing the region containing the gene cassette. The expression of each antigen in vector-infected cells was confirmed by immunoblot analysis using a specific antibody. The vectors were purified from BHH-F5-infected cells by cesium chloride-gradient centrifugation and titrated on BHH-F5 cells by plaque assay to determine the number of PFU per milliliter. (see Sayedahmed et al. (2019), supra, and van Olphen et al., Characterization of bovine adenovirus type 3 E1 proteins and isolation of E1-expressing cell lines, Virology 295: 108-118 (2002)). Vectors having similar VP: PFU ratios were used for immunization studies.

### Example 9

### Absence of BAd3 Cross-Neutralizing Antibodies in Humans

It has previously been demonstrated that approximately 95% of human serum samples have HAd5-neutralizing antibodies, but not none of the samples had BAd3 cross-neutralizing antibodies. (see, e.g., Bangari et al., Comparative transduction efficiencies of human and nonhuman adenoviral vectors in human, murine, bovine, and porcine cells in culture, Biochem Biophys Res Commun 327: 960-966 (2005)). To further test this, 60 additional serum samples were collected from healthy individuals. Similar to the previous results, no detectable levels of BAd3 cross-neutralizing antibodies were detected in any of the 60 samples (see Table 1), whereas approximately 60% of the serum samples had detectable levels of HAd5-specific neutralizing antibodies. Similarly, very low titers of cross-neutralizing antibodies against chimpanzee Ad type 7 (chAd7) were also observed in 32% of serum samples.

TABLE 1
Surveillance of neutralizing antibodies in 60 human
serum samples against HAd5, ChAd7, and BAd3.

| | % of serum samples | % of serum samples |
|---|---|---|
| | with titer above 200 | with titer above 4 |
| HAd5 | 23 | 60 |
| ChAd7 | 0 | 32 |
| BAd3 | 0 | 0 |

### Example 10

Generation and Characterization of HAd and BAd Vectors Expressing HA, HA2, HA2+M2e, or NP with AIP-C5

In addition to Matrix Protein 2 (M2e), a few relatively conserved epitopes have been identified within the HA2 portion (HA stem) of HA that could provide protection from heterosubtypic influenza viruses. To fully explore the potential of the immunologically relevant form of the HA2 domain, it was expressed in HAd and BAd vectors with IgE secretory domain or HA1 signal peptide with or without 4XM2e (e.g., HA2+ secretory signal IgE (SEQ ID NO: 8); HA2+Ig3+M2e (SEQ ID NO:10); HA2+HA1 signal peptide (SEQ ID NO: 12); and HA2+HA1 signal peptide+M2e (SEQ ID NO: 14)). All these constructs contained AIP-C5. The gene constructs, HAd vectors, and BAd vectors that were generated are shown in FIG. 9.

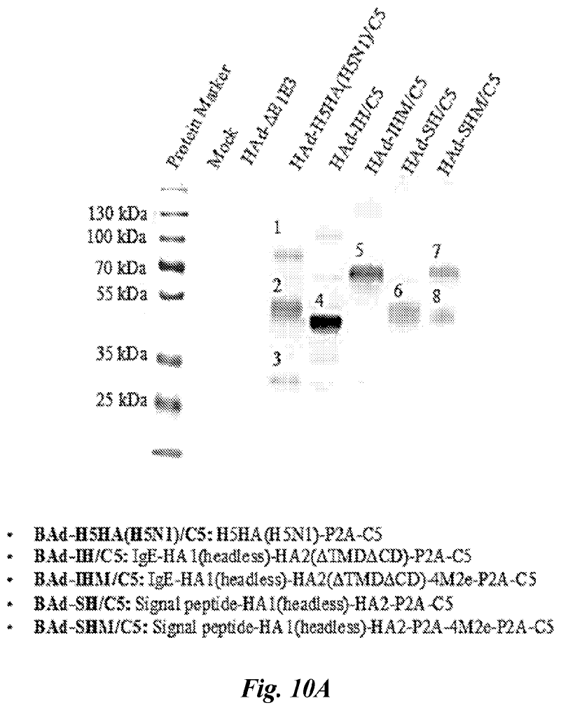

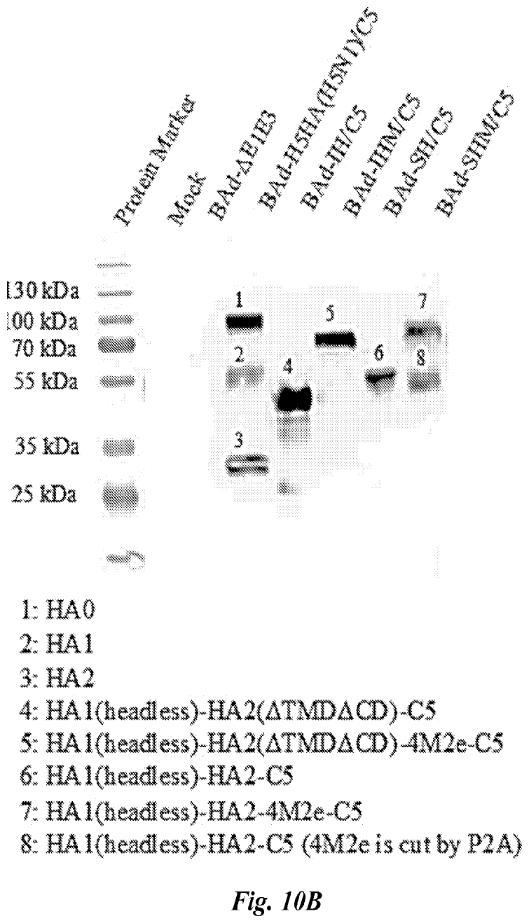

The expression of these gene cassettes in HAd or BAd vectors was confirmed by immunoblotting using H5N1 HA-specific antibody (SEQ ID NO: 6) (FIGS. 10A-10B).

It has been demonstrated that antigen-presenting cells infected with the BAd vector expressing a T cell epitope with AIP-C5 resulted in better antigen presentation to CD4 T cells than the BAd vector expressing only the T cell epitope 22. The impact seemed partly due to autophagy, antigen processing, and lysosomal trafficking. (see Mittal and Jagannath, Novel vaccine formulations for Mycobacterium tuberculosis and use of thereof, U.S. Provisional Patent Application No. 63/160,035 filed 2021; and Khan et al. (2021), supra). It is anticipated that BAd-C5-NP(H7N9) will provide enhanced broad immunity and protection compared to the HAd-C5-NP(H7N9) as observed earlier. (see Sayedahmed et al., A bovine adenoviral vector-based H5N1 influenza-vaccine provides enhanced immunogenicity and protection at a significantly low dose, Mol Ther Methods Clin Dev 10: 210-222 (2018)). BAd-C5-NP(H7N9) and HAd-C5-NP(H7N9) vectors will elicit significantly better immune responses and broad protection as a prime-boost regimen as observed previously. (see Singh et al. (2008), supra).

One of the BAd or HAd vectors expressing HA2+4M2e (FIG. 9) can be used with BAd-C5-NP(H7N9) and/or HAd-C5-NP(H7N9) to further boost heterosubtypic protection against influenza viruses.

### ADDITIONAL CITED REFERENCES

1. Clayville, Influenza update: a review of currently available vaccines, P T 36: 659-684 (2011).
2. Influenza (Seasonal), available online.
3. Sutton, The Pandemic Threat of Emerging H5 and H7 Avian Influenza Viruses, Viruses 10: 461, doi:10.3390/v10090461 (2018).
4. Influenza (Avian and other zoonotic), available online.
5. WHO|Cumulative number of confirmed human cases of avian influenza A(H5N1) reported to WHO, available online.
6. Influenza Type A Viruses|Avian Influenza (Flu), available online.
7. Peacock et al., A Global Perspective on H9N2 Avian Influenza Virus, Viruses 11: 620, doi:10.3390/v11070620 (2019).
8. Belser et al., Infection with highly pathogenic H7 influenza viruses results in an attenuated proinflammatory cytokine and chemokine response early after infection, J Infect Dis 203: 40-48, doi:10.1093/infdis/jiq018 (2011).
9. FAO H7N9 situation update FAO Emergency Prevention System for Animal Health (EMPRES-AH), available online.
10. Sambhara et al., Heterosubtypic immunity against human influenza A viruses, including recently emerged avian H5 and H9 viruses, induced by FLU-ISCOM vaccine in mice requires both cytotoxic T-lymphocyte and macrophage function, Cellular Immun 211: 143-153, doi:10.1006/cimm.2001.1835 (2001).
11. Sambhara et al., Heterotypic protection against influenza by immunostimulating complexes is associated with the induction of cross-reactive cytotoxic T lymphocytes, J Infect Dis 177: 1266-1274 (1998).
12. Berthoud et al., Potent CD8+ T-cell immunogenicity in humans of a novel heterosubtypic influenza A vaccine, MVA-NP+M1, Clin Infect Dis 52: 1-7, doi:10.1093/cid/ciq015 (2011).
13. Rimmelzwaan et al., Influenza virus-specific cytotoxic T lymphocytes: a correlate of protection and a basis for vaccine development, Current Opinion in Biotech 18,: 529-536, doi:10.1016/j.copbio.2007.11.002 (2007).
14. Epstein et al., Protection against multiple influenza A subtypes by vaccination with highly conserved nucleoprotein, Vaccine 23: 5404-5410, doi:10.1016/j.vaccine.2005.04.047 (2005).
15. Del Campo et al., OVX836 a recombinant nucleoprotein vaccine inducing cellular responses and protective efficacy against multiple influenza A subtypes, NPJ Vaccines 4: 4, doi:10.1038/s41541-019-0098-4 (2019).
16. Feng et al., An adenovirus-vectored COVID-19 vaccine confers protection from SARS-COV-2 challenge in rhesus macaques, Nat Commun 11: 4207, doi:10.1038/s41467-020-18077-5 (2020).
17. Wu et al., A single dose of an adenovirus-vectored vaccine provides protection against SARS-CoV-2 challenge, Nat Commun 11: 4081, doi:10.1038/s41467-020-17972-1 (2020).
18. F C et al., Safety, tolerability, and immunogenicity of a recombinant adenovirus type-5 vectored COVID-19 vaccine: a dose-escalation, open-label, non-randomised, first-in-human trial, Lancet 395: doi:10.1016/S0140-6736(20)31208-3 (2020).
19. Logunov et al., Safety and immunogenicity of an rAd26 and rAd5 vector-based heterologous prime-boost COVID-19 vaccine in two formulations: two open, non-randomised phase 1/2 studies from Russia, Lancet 396: 887-897, doi:10.1016/s0140-6736(20)31866-3 (2020).
20. Folegatti et al., Safety and immunogenicity of the ChAd0x1 nCoV-19 vaccine against SARS-CoV-2: a preliminary report of a phase 1/2, single-blind, randomised controlled trial, Lancet 396: 467-478, doi:10.1016/s0140-6736(20)31604-4 (2020).
21. Van Kampen et al., Safety and immunogenicity of adenovirus-vectored nasal and epicutaneous influenza vaccines in humans, Vaccine 23: 1029-1036, doi:10.1016/j.vaccine.2004.07.043 (2005).
22. Jones et al., Prevention of influenza virus shedding and protection from lethal H1N1 challenge using a consensus 2009 H1N1 HA and NA adenovirus vector vaccine, Vaccine 29: 7020-7026, doi:10.1016/j.vaccine.2011.07.073 (2011).
23. Holman et al., Multi-antigen vaccines based on complex adenovirus vectors induce protective immune responses against H5N1 avian influenza viruses, Vaccine 26: 2627-2639, doi:10.1016/j.vaccine.2008.02.053 (2008).
24. Price et al., Vaccination focusing immunity on conserved antigens protects mice and ferrets against virulent H1N1 and H5N1 influenza A viruses, Vaccine 27: doi:10.1016/j.vaccine.2009.08.053 (2009).
25. Kim et al., Mucosal vaccination with recombinant adenovirus encoding nucleoprotein provides potent protection against influenza virus infection, PloS One 8: e75460, doi:10.1371/journal.pone.0075460 (2013).
26. Leung et al., An H5N1-based matrix protein 2 ectodomain tetrameric peptide vaccine provides cross-protection against lethal infection with H7N9 influenza virus, Emerg Microbes Infect 4: e22, doi:10.1038/emi.2015.22 (2015).
27. Staneková and Varečková, Conserved epitopes of influenza A virus inducing protective immunity and their prospects for universal vaccine development, Virol J7: 351, doi:10.1186/1743-422x-7-351 (2010).
28. Vogels et al., Replication-deficient human adenovirus type 35 vectors for gene transfer and vaccination: efficient human cell infection and bypass of preexisting adenovirus immunity, J Virol 77: doi:10.1128/jvi.77.15.8263-8271.2003 (2003).
29. Holterman et al., Novel replication-incompetent vector derived from adenovirus type 11 (Ad11) for vaccination and gene therapy: low seroprevalence and non-cross-reactivity with Ad5. J Virol 78: doi:10.1128/JVI.78.23.13207-13215.2004 (2004).
30. Abbink et al., Comparative seroprevalence and immunogenicity of six rare serotype recombinant adenovirus vaccine vectors from subgroups B and D, J Virol 81: doi:10.1128/JVI.02696-06 (2007).
31. Mittal et al., Development of a bovine adenovirus type 3-based expression vector, J Gen Virol 76(1): 93-102 (1995).
32. Mittal et al., 19 Xenogenic Adenoviral Vectors A2—Curiel, David T, In Adenoviral Vectors for Gene Therapy (2nd Ed.), Academic Press: San Diego: 495-528 (2016).
33. Nachbagauer et al., A chimeric hemagglutinin-based universal influenza virus vaccine approach induces broad and long-lasting immunity in a randomized, placebo-controlled phase I trial, Nat Med 27: 106-114, doi:10.1038/s41591-020-1118-7 (2021).
34. Thomas et al., Cell-mediated protection in influenza infection, Emerg Infect Dis 12: 48-54, doi:10.3201/eid1201.051237 (2006).
35. Zhong et al., Significant impact of sequence variations in the nucleoprotein on CD8 T cell-mediated cross-protection against influenza A virus infections, PloS One 5: e10583, doi:10.1371/journal.pone.0010583 (2010).
36. Roy et al., Partial protection against H5N1 influenza in mice with a single dose of a chimpanzee adenovirus vector expressing nucleoprotein, Vaccine 25: 6845-6851, doi:10.1016/j.vaccine.2007.07.035 (2007).
37. Li et al., Single-dose vaccination of a recombinant parainfluenza virus 5 expressing NP from H5N1 virus provides broad immunity against influenza A viruses, J Virol 87: 5985-5993, doi:10.1128/jvi.00120-13 (2013).
38. McMahon et al., Vaccination with viral vectors expressing chimeric hemagglutinin, NP and M1 antigens protects ferrets against influenza virus challenge, Frontiers in Immun 10: 2005, doi:10.3389/fimmu.2019.02005 (2019).
39. Zhou et al., A universal influenza A vaccine based on adenovirus expressing matrix-2 ectodomain and nucleoprotein protects mice from lethal challenge, Molecular therapy: The Journal of the American Society of Gene Therapy 18, doi:10.1038/mt.2010.202 (2010).
40. Wang et al., Improving Cross-Protection against Influenza Virus Using Recombinant Vaccinia Vaccine Expressing NP and M2 Ectodomain Tandem Repeats, Virologica Sinica 34: 583-591, doi:10.1007/s12250-019-00138-9 (2019).
41. Jagannath et al., Autophagy enhances the efficacy of BCG vaccine by increasing peptide presentation in mouse dendritic cells, Nat Med 15: 267-276 (2009).
42. Lee et al., In vivo requirement for Atg5 in antigen presentation by dendritic cells, Immunity 32: 227-239 (2010).
43. Segura and Amigorena, Cross-presentation in mouse and human dendritic cells, Adv Immunol 127: 1-31, doi:10.1016/bs.ai.2015.03.002 (2015).
44. Amoah et al., Frequency and quality of influenza NP-CD8+ T cells are both critical for heterosubtypic immunity, Under review (2020).
45. Khan et al., A novel bovine adenoviral mucosal vaccine expressing a Mycobacterium tuberculosis antigen-85B epitope and an autophagy-inducing peptide protects mice against tuberculosis through robust pulmonary and systemic immune responses, Cell Rep Med 2: 100372 (2021).
46. Sayedahmed et al., A bovine adenoviral vector-based H5N1 influenza-vaccine provides enhanced immunogenicity and protection at a significantly low dose, Mol Ther Methods Cin Dev 10: 210-222 (2018).

We claim:

1. An immunogenic composition comprising:
a full-length nucleoprotein (NP) of a H7N9 influenza virus with or without expressing 22 amino acid residues of Autophagy-Inducing Peptide C5 (AIP-C5) from a CFP10 protein of Mycobacterium tuberculosis, or a functional fragment thereof, and
a pharmaceutically acceptable carrier;
wherein the immunogenic composition is cross-protective against two or more subtypes of influenza viruses when administered to a subject.

2. The immunogenic composition of claim 1, wherein the composition is cross-protective against at least five subtypes of influenza viruses when administered to a subject.

3. The immunogenic composition of claim 1 or 2, wherein, when administered to a subject, the composition confers general immunogenicity protection against the subtypes of viruses selected from the group consisting of H1, H3, H5, H7, H9 and influenza B viruses.

4. The immunogenic composition of claim 1, wherein the composition is cross-protective against two or more subtypes of influenza A or B viruses when administered to a subject.

5. The immunogenic composition of claim 1, wherein the full-length NP or functional fragment thereof comprises SEQ ID NO: 1.

6. The immunogenic composition of claim 1 or 5, wherein the AIP-C5 from a CFP10 protein comprises SEQ ID NO: 3.

7. The immunogenic composition of any one of claims 1, 2, 3, 4, 5, or 6, further comprising an adjuvant.

8. The immunogenic composition of any one of claims 1, 2, 3, 4, 5, or 6 formulated to be administered intranasally.

9. The immunogenic composition of any one of claims 1, 2, 3, 4, 5, or 6 formulated to be administered subcutaneously.

10. The immunogenic composition of any one of claims 1, 2, 3, 4, 5, or 6 formulated for oral administration.

11. The immunogenic composition of any one of claims 1, 2, 3, 4, 5, or 6 formulated as an aerosol spray.

12. An immunogenic composition comprising:
SEQ ID NO: 6, 8, 10, 12, or 14; and
a pharmaceutically acceptable carrier;
wherein the immunogenic composition is cross-protective against two or more subtypes of influenza viruses when administered to a subject.

13. A human or bovine adenoviral (Ad) vector comprising a polynucleotide sequence that encodes a full-length nucleoprotein (NP) from H7N9 influenza virus, a functional fragment thereof and/or one or more other immunogenic domains of an influenza virus.

14. The Ad vector of claim 13, wherein the polynucleotide sequence further encodes Autophagy-Inducing Peptide C5 (AIP-C5) from the CFP10 protein of Mycobacterium tuberculosis.

15. The Ad vector of claim 14, wherein the AIP-C5 comprises 22 amino acid residues.

16. The Ad vector of claim 13, wherein the polynucleotide sequence comprises SEQ ID NO: 2.

17. The Ad vector of any one of claim 12, 13, or 14, wherein the polynucleotide sequence further comprises SEQ ID NO: 4.

18. The Ad vector of claim 13, wherein the full-length NP, functional fragment thereof and/or one or more other immunogenic domains of an influenza virus comprises SEQ ID NO: 1.

19. The Ad vector of claim 13, wherein the AIP-C5 comprises SEQ ID NO: 3.

20. The Ad vector of claim 13, wherein at least E1 and E3 regions are deleted.

21. The Ad vector of claim 20, wherein the polynucleotide sequence of the full-length NP, functional fragment thereof and/or one or more other immunogenic domains of an influenza virus is inserted in the deleted E1 region.

22. The Ad vector of claim 19, wherein at least E1 and E3 regions of the Ad are deleted and SEQ ID NO: 3 is inserted in the deleted E1 region of the Ad.

23. The Ad vector of claim 13, wherein said Ad provides protection against infection by various subtypes of viruses selected from the group consisting of H1, H3, H5, H7, H9, and influenza B viruses.

24. The Ad vector of claim 13, wherein the Ad is bovine Ad type 3 (BAd3).

25. A human or bovine adenoviral (Ad) vector comprising a polynucleotide sequence comprising SEQ ID NO: 5, 7, 9, 11, 13, or 15, or a functional fragment thereof.

26. A method of generating a general immunogenicity against a heterosubtypic influenza virus in a subject, comprising administering to said subject an effective amount of an immunogenic composition of any one of claims 1-13 or an adenoviral (Ad) vector of any one of claims 12-25.

27. The method of claim 26, wherein said administration is intranasal.

28. The method of claim 26, wherein said administration is subcutaneous.

29. The method of claim 26, wherein said composition or Ad vector is administered orally.

30. The method of claim 29, wherein said composition or Ad vector is administered as an aerosol spray.

31. The method of claim 26, wherein said method provides a general immunogenicity protection to the subject against various subtypes of viruses selected from the group consisting of H1, H3, H5, H7, H9, and influenza B viruses.

32. The method of claim 26, wherein administration of the effective amount of the immunogenic composition or the Ad vector induces a dose-dependent increase in cell-mediated immunity in the subject.

33. The method of claim 26, wherein the subject is a human.

34. The method of claim 26, wherein said administration is intramuscular.


SEQUENCE LISTING (15 sequences)

SEQ ID NO: 1 - protein, 498 aa, Influenza A virus
  source 1..498: note = subtype H7N9
MASQGTKRSYEQMETGGERQNATEIRASVGRMVSGIGRFYIQMCTELKLSDNEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGIGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRMVPRGQLSTRGVQIASNENMEAMDSNTLELRSRYWAIRTRSGGNTNQQRASAGQVSVQPTFSVQRNLPFERATIMAAFTGNTEGRTSDMRTEIIRMMESARPEDVSFQGRGVFELSDEKATNPIVPSFDMNNEGSYFFGDNAEEYDN

SEQ ID NO: 2 - DNA, 1497 bp, Influenza A virus
  source 1..1497: mol_type = genomic DNA; note = subtype H7N9
atggcttcccagggcacaaagaggtcttacgagcagatggagaccggcggagagagacagaacgccacagagatcagagctagcgtgggacggatggtgtccggaatcggccgcttctacatccagatgtgcaccgagctgaagctgtccgacaacgagggccggctgatccagaactccatcacaatcgagcgcatggtgctgtctgcctttgacgagaggagaaacagatacctggaggagcacccttctgctggaaaggatccaaagaagaccggaggaccaatctaccggcgcagggacggcaagtgggtgagagagctgatcctgtacgataaggaggagatcagacggatctggcggcaggccaacaacggagaggacgccaccgctggcctgacacacctgatgatctggcacagcaacctgaacgacgccacctaccagcgcacaagggctctggtgaggaccggaatggatcccagaatgtgctccctgatgcagggctctacactgcctcgcaggtccggagctgctggagctgctgtgaagggaatcggcaccatggtcatggagctgatcagaatgatcaagcggggcatcaacgatcgcaacttctggaggggagagaacggcagacggacccgcatcgcctacgagaggatgtgcaacatcctgaagggcaagtttcagacagccgctcagagggccatgatggaccaggtgagagagtcccggaaccccggaaacgctgagatcgaggatctgatcttcctggctcggtctgctctgatcctgaggggaagcgtggctcacaagtcctgcctgccagcttgcgtgtacggactggccgtggcttctggctacgactttgagcgggagggatacagcctggtgggcatcgatcccttccgcctgctgcagaactctcaggtgtttagcctgatcaggccaaacgagaaccccgcccacaagagccagctggtgtggatggcttgtcactccgccgctttcgaggacctgcgggtgagctcctttatccgcggcaccaggatggtgcctaggggacagctgagcacaagaggcgtgcagatcgcctccaacgagaacatggaggctatggattctaacaccctggagctgagaagccggtactgggctatcaggaccaggagcggcggaaacacaaaccagcagagggcttctgctggacaggtgagcgtgcagcctaccttctccgtgcagcggaacctgccatttgagcgcgccacaatcatggccgctttcaccggaaacacagagggcagaacctctgacatgcggacagagatcatccgcatgatggagtccgccaggccagaggacgtgagcttccagggaagaggcgtgtttgagctgagcgacgagaaggctacaaaccccatcgtgccttctttcgatatgaacaacgagggaagctacttctttggcgacaacgccgaggagtacgataactga

SEQ ID NO: 3 - protein, 22 aa, Mycobacterium tuberculosis
MAAQAAVVRFQEAANKQKQELD

SEQ ID NO: 4 - DNA, 66 bp, Mycobacterium tuberculosis
  source 1..66: mol_type = genomic DNA
atggccgctcaggccgctgtggtgagattccaggaggccgctaacaagcagaagcaggagctggac

SEQ ID NO: 5 - protein, 9 aa, synthetic construct
TYQRTRALV

SEQ ID NO: 6 - protein, 613 aa, synthetic construct
  SIGNAL 1..16
  REGION 17..346
  REGION 347..568
  PEPTIDE 574..613
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICIGPGPGATNFSLLKQAGDVEENPGPAAQAAVVRFQEAANKQKQELD

SEQ ID NO: 7 - DNA, 1842 bp, synthetic construct
  source 1..1842: mol_type = other DNA
atggagaaaatagtacttctctttgctatcgttagcctggtcaagtctgaccagatctgcatcggctaccatgctaacaacagtactgagcaagttgatactattatggagaagaatgttactgtcacacatgctcaggacatcctggaaaagaagcacaacgggaagctgtgcgatctggatggtgtgaagcctctcattcttagagactgctcagtggccggttggctcctaggcaaccctatgtgtgatgagttcatcaacgttccggagtggagctatatcgtggaaaaagctaatccagtaaatgacctttgctaccctggtgactttaacgattatgaagaactaaaacacctgctgagtcggattaaccactttgagaaaatccagattatccctaagagcagctggtcatctcatgaggccagcctgggagtctcctcagcctgtccataccaagggaaatcttccttcttccggaatgtggtctggctgatcaaaaaaaattcaacgtaccccacaataaagagatcctacaacaatactaatcaagaagacttactcgtgctttggggaattcaccatcccaacgatgccgctgagcagaccaaactgtatcagaaccccaccacttacatcagcgtgggcaccagcaccttgaaccagcgccttgtcccgcgcattgccacaaggtctaaggtaaacggacagtcaggcaggatggaatttttctggacaatactgaagccaaatgatgccataaactttgaatccaatggcaattttatcgctccagagtatgcctacaaaatagtaaaaaaaggagactcaactataatgaagtcagaactagaatatggaaactgcaacaccaagtgccagacacccatgggtgctatcaattcgtccatgcctttccataacatccacccactgacgattggggagtgtcccaagtatgtgaagagtaaccggttggtgctcgccaccggacttcgaaattcccctcagagagaacgtcgtcggaagaaacgcgggctgtttggagcaatcgctggcttcattgaagggggatggcagggtatggtggatggctggtacggctatcaccacagcaacgaacagggctctggatacgcagcagacaaggagtctacccagaaggcgattgatggggtcaccaataaagtcaactcgatcattgacaaaatgaatacacagtttgaagcagtaggtcgcgagtttaataatttggaaaggcgaattgagaatcttaacaagaagatggaggatggctttttggatgtgtggacctacaatgcggagctcctcgtgctgatggaaaatgagcgaacgctggacttccatgactctaacgtgaaaaacctctacgataaggttcggctccaactcagagacaatgcgaaggaactggggaacggctgcttcgagttctaccacaagtgtgacaatgaatgcatggagagcgtcagaaatggcacatatgactatccacagtacagcgaggaggcaagattgaagagggaggaaatttctggggttaagctggagtccattggaatctaccagatcttaagtatctatagtactgtggccagttctctggccttggctatcatggtcgcagggttgtcgctatggatgtgcagcaacggctcgctgcagtgtaggatttgtattggtcctgggcccggagctacaaacttcagcctcctgaagcaggccggcgacgtggaggagaaccccgggccagcagcccaggcagctgtggtgaggttccaggaggccgccaacaaacaaaaacaagagctggattga

SEQ ID NO: 8 - protein, 353 aa, synthetic construct
  SIGNAL 1..19
  REGION 20..61
  REGION 66..122
  REGION 123..308
  PEPTIDE 314..353
MKLPVRLLVLMFWIPASSSDQICIGYHANNSTEQVDTIMEKNVTVTHAQDILEKKHNGKLCGGGGCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQIGPGPGATNFSLLKQAGDVEENPGPAAQAAVVRFQEAANKQKQELD

SEQ ID NO: 9 - DNA, 1062 bp, synthetic construct
  source 1..1062: mol_type = other DNA
atgaaactgccagtccgactccttgttttgatgttctggatacctgccagctcttcagaccagatctgcattggctaccatgcgaacaattctacagagcaggtggacaccatcatggaaaagaatgtaactgtgacacatgctcaagacatcctggagaagaagcacaatgggaaactctgtgggggaggtgggtgtaataccaagtgccaaacgcccatgggggccatcaacagttcgatgcccttccacaacatccatcccctgacaataggagagtgtcccaagtatgtgaaatccaacagattggttctggctactggcttgcggaacagccctcaaagagaaagacgccggaaaaagaggggcctttttggggccattgctggcttcattgaaggtggctggcagggcatggtggatggatggtacggctatcaccactccaatgaacaaggttctggttatgctgctgataaagaatccacacagaaggccattgatggagtcactaacaaagtcaacagcattatcgataagatgaacacccagtttgaggcagtgggccgggaattcaacaacctggaacgacgtattgaaaacttaaacaagaaaatggaggatgggtttctggatgtgtggacatacaacgcagagctcctagtgcttatggaaaatgagaggactctggactttcatgattcaaatgttaaaaatctttatgacaaagtccgcctacagctcagggacaatgccaaggagctggggaatggctgctttgagttctaccacaagtgtgacaacgagtgcatggagagtgtcagaaatggaacctatgattacccacagtacagtgaagaggccaggctgaagcgggaggaaatctctggagtgaagctggagtccatcggtatataccagattggaccgggacctggagcgaccaacttcagcctgttgaagcaagcaggggacgtagaggagaaccctggcccagcagcccaggctgctgtggttcgcttccaggaagcagccaataagcagaaacaggaattagactga

SEQ ID NO: 10 - protein, 465 aa, synthetic construct
  SIGNAL 1..19
  REGION 20..61
  REGION 66..122
  REGION 123..308
  REGION 314..336
  REGION 342..364
  REGION 370..392
  REGION 398..420
  PEPTIDE 426..465
MKLPVRLLVLMFWIPASSSDQICIGYHANNSTEQVDTIMEKNVTVTHAQDILEKKHNGKLCGGGGCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQIGPGPGSLLTEVETPTRNEWECRCSDSSDGPGPGSLLTEVETPTRTGWECNCSGSSEGPGPGSLLTEVETPTRNEWECRCSDSSDGPGPGSLLTEVETPTRTGWECNCSGSSEGPGPGATNFSLLKQAGDVEENPGPAAQAAVVRFQEAANKQKQELD

SEQ ID NO: 11 - DNA, 1398 bp, synthetic construct
  source 1..1398: mol_type = other DNA
atgaagctaccagtcagactattggtgctgatgttctggattcctgcgagcagttctgaccagatctgtatcggctaccatgcaaacaacagcacagagcaggttgataccatcatggagaagaacgtcacagtgacacatgcccaggacatcctggagaagaagcacaatggaaaactgtgtggtggtgggggatgcaacacaaagtgccagacccccatgggagcgattaattcctccatgccttttcacaacatccatcctctcaccattggtgaatgtcccaaatatgttaaatcgaataggctcgtactggccacagggttaaggaattcaccacagcgggagagacggaggaagaagaggggactctttggggcaattgctggcttcatcgaaggcggctggcagggcatggtggatggatggtatggataccaccacagtaacgagcaaggaagcggctatgctgctgacaaagaaagcacccagaaagccattgatggagtcaccaacaaggtgaattctataatagacaagatgaacacacagtttgaggcagttggtcgggagtttaataacctggagcgccgcattgagaatctgaataaaaaaatggaggacggattcctggatgtctggacctacaacgcagagttgcttgttctcatggaaaatgagcggaccctggactttcatgactctaatgtgaagaacctgtatgataaagtgaggctgcaattgagagataatgctaaggagcttggaaatggctgctttgaattctaccacaagtgtgataatgagtgcatggaatctgtgcgaaacggcacctatgactacccgcagtactccgaagaagcccgtctgaagcgagaagaaatcagtggtgtcaaactggagagcatagggatctaccagattggccccgggccgggatctctccttactgaagtagagaccccaaccagaaatgaatgggagtgccgctgcagcgattcgtcagacggcccaggtcccgggtccttgctgacagaagttgaaacacccacgcgaacgggttgggaatgtaactgttccgggtcctcagaagggcctgggcctggttctctactgactgaggtggaaactcccactcgtaacgagtgggaatgcagatgtagtgactccagcgacgggccaggtccaggatcactcttgacagaggtagagactcccactagaactggctgggagtgtaactgcagtggcagttctgaagggccaggccctggcgccacgaacttctcccttttaaagcaggccggagatgtggaggagaacccagggcctgctgctcaagcagctgtggtcaggttccaggaggccgccaacaaacagaagcaagagctggattga

SEQ ID NO: 12 - protein, 382 aa, synthetic construct
  REGION 13..58
  SIGNAL 1..12
  REGION 63..119
  REGION 120..341
  PEPTIDE 347..382
MEKIVLLFAIVSLVKSDQICIGYHANNSTEQVDTIMEKNVTVTHAQDILEKKHNGKLCGGGGCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICIGPGPGATNFSLLKQAGDVEENPGPAAQAAVVRFQEAANKQK

SEQ ID NO: 13 - DNA, 1149 bp, synthetic construct
  source 1..1149: mol_type = other DNA
atggagaaaattgtgctactgtttgctatagtcagtctggtgaaatctgaccagatctgtattggctaccatgccaacaactccacagaacaggtcgacactataatggaaaagaatgttacggtgacacacgcccaggacatcttggagaagaagcacaatgggaagttgtgtgggggaggtggatgcaacaccaagtgccagacccccatgggcgccatcaattcatccatgcccttccacaacatacaccctctcaccatcggagaatgtccaaaatatgtgaagtccaaccgtctcgtcttggcaactggtcttcggaacagtccacagagagaacgccgccgcaaaaagagaggtctttttggagctattgctggtttcatcgaaggcggctggcagggcatggttgatggatggtacgggtatcatcatagcaacgagcaaggctctggatatgcagcagacaaagaatctactcagaaggccattgatggtgtcaccaacaaggtgaacagcatcatagacaagatgaacacacagtttgaagctgtgggccgggagtttaacaatctcgaacgaaggattgagaacctgaataagaaaatggaagatgggttcctggatgtctggacatacaacgctgagctccttgttctgatggagaatgagcggacgctggacttccatgactcgaatgttaaaaatttgtatgataaagtaaggctgcagctgagggacaatgccaaggagctaggcaacggctgctttgagttctaccacaagtgcgataatgaatgcatggagtccgtcagaaatggaacctatgactaccctcaatatagtgaagaggcccgattaaaaagggaggagatctccggcgtaaagctggagagcattggaatttaccagattctttcaatctacagtactgtggcctcttctctggccctggctatcatggtggctgggctaagtctctggatgtgtagcaatggtagcttacagtgtagaatctgcatcgggcccgggcctggcgcgacaaacttctcattgctgaagcaggctggagatgtggaggaaaacccagggccggccgcgcaagcagcagtggtacgtttccaggaagcagccaataaacaaaaatga

SEQ ID NO: 14 - protein, 517 aa, synthetic construct
  SIGNAL 1..16
  REGION 17..58
  REGION 62..119
  REGION 120..341
  REGION 347..365
  REGION 366..388
  REGION 394..416
  REGION 422..444
  REGION 450..472
  PEPTIDE 478..517
MEKIVLLFAIVSLVKSDQICIGYHANNSTEQVDTIMEKNVTVTHAQDILEKKHNGKLCGGGGCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICIGPGPGATNFSLLKQAGDVEENPGPSLLTEVETPTRNEWECRCSDSSDGPGPGSLLTEVETPTRTGWECNCSGSSEGPGPGSLLTEVETPTRNEWECRCSDSSDGPGPGSLLTEVETPTRTGWECNCSGSSEGPGPGATNFSLLKQAGDVEENPGPAAQAAVVRFQEAANKQKQELD

SEQ ID NO: 15 - DNA, 1554 bp, synthetic construct
  source 1..1554: mol_type = other DNA
atggagaagatcgtactgctttttgccatagttagcttggtcaagtctgaccagatctgcatcggctaccatgcaaacaatagcaccgagcaagtggacaccataatggagaaaaatgtgactgtgacacatgcccaggacatcttagaaaagaaacacaatgggaagctctgtgggggagggggctgtaataccaagtgccagacccccatgggcgccatcaacagctccatgcctttccacaacattcaccccctgacaattggggagtgtcccaaatatgtgaaaagcaacagactggtccttgcgactggcctacgtaactctccacaacgggagcggagaaggaaaaagagaggtctttttggagcaattgccggattcattgaaggcggctggcagggaatggtggatggatggtacggttatcatcactctaacgagcagggctccgggtatgcagctgacaaggagtccactcaaaaggccattgatggtgttaccaacaaagtgaattcaataattgacaaaatgaacactcagtttgaggctgttggtcgcgagttcaacaacctggagcgtagaatagagaatctgaacaagaagatggaggacggcttcctggacgtgtggacctacaacgctgagttgctcgtcctgatggaaaacgaaaggaccttggactttcatgacagcaatgtaaagaacctctatgataaggtccggctccagctgcgggataatgccaaagagctgggcaacggctgctttgaattctaccacaagtgtgacaatgaatgcatggaaagtgttcgcaatggcacttatgactacccacagtacagtgaagaagctcgacttaaaagagaagagatcagcggagtgaagctggaaagcattggaatctaccagatcctatccatctattccacagtcgcctcttcactagctttggccatcatggtagcaggtctgagcctctggatgtgctcgaatggaagtctgcagtgcaggatctgtattggaccaggaccgggtgcaacaaacttctccctcctgaagcaggccggcgatgtcgaggagaacccggggccttcgctgctcacagaagtggaaactcctacacgcaacgagtgggaatgtcgatgctcagattctagtgacggtcccggaccaggcagccttctgacggaagtagagacaccaacaaggactggttgggaatgtaactgttcaggcagcagcgaggggcctggacctggttctttgctgacggaggtggagacacctaccaggaatgaatgggagtgccgctgttctgattcttcagatgggccaggcccgggttcattactcaccgaggttgaaacgcccacccggacaggctgggagtgcaactgcagtggcagcagcgaagggccagggcccggggccaccaatttttccttactgaaacaagctggagatgtggaggaaaatcctgggcccgcagcacaggctgctgtggtcagattccaggaggccgcgaataaacagaagcaagaattagattga